(19) United States
(12) Patent Application Publication
(10) Pub. No.: US 2014/0018256 A1
(43) Pub. Date: Jan. 16, 2014

(54) METHOD OF DIAGNOSING AUTISM SPECTRUM DISORDER

(71) Applicants: Patrick Johnston, London (GB); David Hardoon, Singapore (SG); Declan Murphy, London (GB); John Powell, London (GB); Christine Ecker, London (GB)

(72) Inventors: Patrick Johnston, London (GB); David Hardoon, Singapore (SG); Declan Murphy, London (GB); John Powell, London (GB); Christine Ecker, London (GB)

(73) Assignees: King's College London, London, EN (GB); David Hardoon, Singapore (SG); Patrick Johnston, London, EN (GB)

(21) Appl. No.: 13/981,244

(22) PCT Filed: Jan. 23, 2012
(86) PCT No.: PCT/GB2012/050135

§ 371 Date: Oct. 3, 2013

(30) Foreign Application Priority Data
Jan. 24, 2011 (GB) 1101200.2

(51) Int. Cl. C12Q 1/68

(52) U.S. Cl. CPC C12Q 1/6883
USPC 506/9; 506/16; 506/30

(57) ABSTRACT

The present invention provides a method for diagnosing an autism spectrum disorder (ASD), or predisposition to develop an ASD, in a subject, which comprises the step of investigating a set of single nucleotide polymorphisms (SNPs) in a sample from the subject, wherein the number of SNPs in the set is such that the method can diagnose ASD with at least 70% accuracy. The invention also provides a kit for diagnosing an ASD, or predisposition to develop an ASD, in a subject, which comprises a plurality of primer pairs or probes capable of investigating such a set of SNPs in a sample from a subject, and a method for making such a kit.

### FIELD OF THE INVENTION

The present invention relates to the diagnosis of Autism spectrum disorder (ASD), or predisposition to develop ASD. In particular it relates to a method for diagnosing an ASD, or predisposition to develop an ASD, by investigating a set of single nucleotide polymorphisms (SNPs) in a sample from a subject.

### BACKGROUND TO THE INVENTION

Autism Spectrum Disorders (ASDs) are a spectrum of neurodevelopmental conditions characterized by impairments in social interaction and communication, and associated with repetitive, restricted patterns of interest or behaviour. Autism Spectrum Disorders is an umbrella term used to describe a number of autism disorders such as classic autism, Asperger's Syndrome, atypical autism and pervassive developmental disorder not otherwise specified.

ASDs are relatively common neurodevelopmental disorders, affecting approximately 1% of the population. Autism shows a well established gender distortion with about four times as many males as females being affected.

Currently a diagnosis of ASD is formulated using behavioural criteria, with the aid of diagnostic manuals, for example the International Classification of Disease (ICD-10) and Diagnostic and Statistic Manual of mental health (DSM-IV). The diagnosis of autism is not unified and a number of distinct criteria are applied in different parts of the world. In many European countries diagnostic criteria like DSM-IV for psychiatric diseases are applied. The Autism Diagnostic Interview (ADI) and Autism Diagnostic Observation Schedule (ADOS) are diagnostic tests, and have become a kind of ‘gold standard’ and are increasingly being implemented in both the USA and Europe.

The Autism Diagnostic Interview-Revised (ADI-R) is a diagnostic assessment for ASD. It is a parental interview that probes for language, social, behavioural, and functional abnormalities that are inconsistent with a specific child's stage of development. The ADI-R is a standardized, semi-structured clinical review for caregivers of children and adults. The interview contains 111 items and focuses on behaviours in three content areas: quality of social interaction, (e.g., emotional sharing, offering and seeking comfort, social smiling and responding to other children); communication and language (e.g., stereotyped utterances, pronoun reversal, social usage of language); and repetitive, restricted and stereotyped interests and behaviour (e.g., unusual preoccupations, hand and finger mannerisms, unusual sensory interests). Responses are scored by the clinician based on the caregiver's description of the child's behaviour. This interviewer-based instrument requires substantial training in administration and scoring, making it very time-consuming and expensive. As diagnosis also depends on the assessment of both the caregiver and interviewer, it is also highly subjective.

The main treatment proposed for ASDs are based on intensive educational programs, but also include pharmacotherapy and cognitive behavioural approaches. Sustained special education programs and behavior therapy early in life can help children acquire self-care, social, and job skills. Available approaches include applied behavior analysis (ABA), developmental models, structured teaching, speech and language therapy, social skills therapy, and occupational therapy. Applied early enough, studies have shown that as many as 50% of autistic children participating in such programs can be referred back to normal schooling and education. In a recent UK study the potential socio-economic benefit of early intensive treatment has been estimated to be as high as £1.8 million per patient over the life-time of the patient.

However, the age at which the therapy is provided and started is of significant importance. Ideally, it is thought that the programs should start at 18 months age, at the latest.

As outlined above, the ADI-R cannot be used for diagnosis under the age of 18 months. Indeed, for infra-structural (availability of trained experts, in the US only 10% of suspected autistic children have direct access to specialists able to carry out ADI-R) and social reasons the average age of diagnosis is 5 years in the US and 8 years in some parts of Europe.

Of importance also is that there is increasing recognition that many adults with ASD have not been recognised or diagnosed. In adults, however, current symptoms are often modulated by coping strategies developed over the life-span, and retrospective accounts of past symptoms rely not only on the availability of an informant but also on their reliability. Moreover, for reasons of confidentiality, many adults do not wish others to be interviewed about their condition—and so a full diagnostic developmental history cannot be obtained to allow a confident diagnosis of ASD.

Hence there is a clear need for improved diagnostic methods for ASDs which address the problems associated with the behavioural characterisation studies currently in use. In particular, there is a need for an early-detection method enabling early intervention, which is thought to be essential in order to have a significant impact on the child's development.

### DESCRIPTION OF THE FIGURES

FIG. 1—A graph to show the relationship between the number of SNPs used in the diagnostic assay and the overall accuracy of ASD affected/unaffected classification.

### SUMMARY OF THE INVENTION

The present inventors have developed a new genetic test which can diagnose ASD with over 96% accuracy. The advantage of a genetic test is that it can be conducted at any age, for example at birth or during babyhood, allowing appropriate educational programmes to be started in early infancy and maximal benefit to be gained from such programs. It can also be applied in adulthood. A genetic test also does away with the need for trained professional to carry out behavioural testing, and addresses the problems associated with inconsistencies between the different behavioural tests and subjectivity of the caregiver/interviewer.

Thus, in a first aspect, the present invention provides a method for diagnosing an autism spectrum disorder (ASD), or predisposition to develop an ASD, in a subject, which comprises the step of investigating a set of single nucleotide polymorphisms (SNPs) in a sample from the subject, wherein the number of SNPs in the set is such that the method can diagnose ASD with at least 70% accuracy.

The set of SNPs may be at least partly derivable from the list of SNPs given in Table 3. For example, the set may comprise at least 1500 SNPs, at least 2300 SNPs or all 3126 SNPs from the list given in Table 3.

The set of SNPs may comprise at least 70% of the SNPs weighted at least ±0.01 in Table 3.

The set of SNPs may comprise between 1500 and 4500 SNPs, between 2300 and 3900 SNPs, or between 3000 and 3300 SNPs.

The set of SNPs may comprise one or more SNPs which are highly correlated with one or more SNPs from the list given in Table 3.

In a second aspect, the present invention provides a kit for diagnosing an autism spectrum disorder (ASD), or predisposition to develop an ASD, in a subject, which comprises a plurality of primer pairs or probes capable of investigating a set of single nucleotide polymorphisms (SNPs) in a sample from the subject, wherein the set of SNPs is as defined in accordance with the first aspect of the invention.

Where the kit comprises a plurality of probes, they may be immobilised on a solid support.

In a third aspect, the present invention provides a method for preparing a kit according to the second aspect of the invention which comprises the step of immobilising the plurality of probes on to a solid support.

### DETAILED DESCRIPTION

### Autism Spectrum Disorder

Autism spectrum disorders (ASD) are developmental disorders resulting from dysfunction in the central nervous system and are characterized by impairments in three behavioural areas: communication (including spoken language), social interactions, and repetitive behaviours or restricted interests. ASDs usually manifest before three years of age and the severity can vary greatly. Idiopathic ASDs currently include autism, which is considered to be the most severe form; pervasive developmental disorders not otherwise specified (PDD-NOS); and Asperger's syndrome, a form of autism in which persons can have relatively normal intelligence and communication skills but difficulty with social interactions.

ASD may be diagnosed using behavioural criteria, with the aid of diagnostic manuals, for example the International Classification of Disease (ICD-10) and Diagnostic and Statistic Manual of mental health (DSM-IV); or by using Autism Diagnostic Interview-Revised (ADI-R) which is a diagnostic assessment for ASD.

The method of the present invention may be capable of diagnosing an autism spectrum disorder (ASD), for example it may be used to establish or confirm that a subject is affected by an ASD. In this embodiment, the subject may already show symptoms of the ASD, such as impaired social interaction and/or communication, or repetitive patterns of interest or behaviour.

The method of the present invention may be capable of diagnosing or detecting a predisposition to develop an ASD. For example, it may be used to predict the likelihood that a subject will develop an ASD, maybe before the subject shows one or more symptom(s) of an ASD. This embodiment is particularly useful for the evaluating the likelihood of ASD development in a subject too young for ASD examination using classical behavioural analysis, such as a subject less that 18 months old. Also it will allow diagnosis in people (e.g. adults or refugees) who have no informants available to confirm their developmental history.

### Single Nucleotide Polymorphisms

As used herein, single-nucleotide polymorphism (SNP) is a DNA sequence variation occurring when a single nucleotide (A, T, C, or G) in the genome differs between an individual affected with an ASD and an unaffected individual.

Single nucleotides may be changed (substitution), removed (deletions) or added (insertion) to a polynucleotide sequence. Insertion or deletion SNPs (InDels) may shift translational frame.

Single nucleotide polymorphisms may fall within coding sequences of genes, non-coding regions of genes, or in the intergenic regions between genes.

The method of the invention involves investigating a set of single nucleotide polymorphisms (SNPs) in a sample from the subject. In other words, the presence of absence of a plurality of SNPs in a subject is analysed, in order to give an overall “score” from which it can be deduced whether a subject has, or is likely to develop a predisposition to ASD.

SNPs may be defined by their position within the genome, for example as an “rs” number (see Table 3). Relevant sequence information may be found from public databases such as http://genome.uscs.edu or http://www.ncbi.nlm.nih.gov/snp.

### SNP Sets

Using the Autism Genetic Resource Exchange (AGRE) sample (see below), comprising 1385 individuals with ASD and 1494 unaffected individuals, a whole genome association study identified 390671 SNPs.

Subsequent analysis revealed that accuracy could be improved by using a sub-set of this total number, including the most “influential” SNPs (i.e. those with the highest weighting). A SNP set consisting of the 3126 SNPs listed in Table 3 gave an overall ASD classification accuracy of over 96.6%.

It is likely that small modifications to the number of SNPs in the set can be made without significantly affecting accuracy. For example, the nine SNPs from Table 3 shown below:
rs7965985
rs753213
rs7403957
rs4698515
rs3923686
rs6908859
rs7794971
rs793091
SNP_A-1992337
   are listed with a weighting of “0”, so it is likely that any or all of these could be removed without affecting classification accuracy.

It is believed, however that larger increases or decreases in the number of SNPs in the set will decrease accuracy, but this may still be within acceptable levels for a diagnostic test.

For example, as shown in Table 2, a SNP set comprising 2345 SNPs achieved an accuracy of 89% and a SNP set comprising 3907 SNPs achieved an accuracy of 89.09%.

In accordance with the present invention, the number of SNPs in the SNP set should be such that the accuracy of ASD classification is at least 70%.

The SNP set may comprise at least 1500, at least 2300, or at least 3000 SNPs from the list given in Table 3.

The SNP set may comprises substantially all 3126 SNPs given in Table 3. For example, the SNP set may comprise between 3110 and 3126 SNPs from the list given in Table 3.

The SNP set may comprises at least 70%, 80%, 90% or 95% of the SNPs weighted at least ±0.01 in Table 3.

The SNP set may comprises at least 70%, 80%, 90% or 95% of the SNPs weighted at least ±0.02 in Table 3.

The SNP set may comprises at least 70%, 80%, 90% or 95% of the SNPs weighted at least ±0.03 in Table 3.

The SNP set may comprises at least 70%, 80%, 90% or 95% of the SNPs weighted at least ±0.04 in Table 3.

The SNP set may comprise, for example, between 1500 and 4500, between 2300 and 3900 SNPs, or between 3000 and 3300 SNPs.

### Highly Correlated SNPS

The SNP set may comprise on or more SNPs which are highly correlated with one or more SNPs from the list given in Table 3 Linkage disequilibrium (LD) is the measure of how correlated one SNP is to another. Within the list of SNPs given in Table 3, a person skilled in the art can calculate SNPs which will be in high correlation (LD) with those SNPs, which in turn may be predictive for ASDs.

LD provides a score (r2) ranging from 0-1. A highly correlated SNP may have a score of at least 0.7, 0.8 or 0.9 based on the set of SNPs given in Table 3.

### Accuracy

The level of accuracy obtained using a given SNP set may be obtained by challenging the SNP to diagnose ASD for a group of individuals whose ASD status is already known, for example by standard behavioural classification.

The % accuracy for a given SNP set may be obtained by:

number of correctly classified individuals/number of individuals×100

The group of individuals may comprise ASD affected subjects, ASD unaffected subjects or a combination of both types of subject. Where the group comprises both ASD affected and ASD unaffected subjects, the SNP set is challenged for its capacity to identify individuals both “positively” and “negatively”, providing a more robust result.

The group of individuals should be large enough to ensure that the calculated accuracy levels are statistically significant. Too small a test group may not provide a complete picture of the significance of a given result, whether it is a positive or negative correct classification or a “false positive” or “false negative”.

The test group may, for example, comprise at least 100, 500 or 1000 individuals.

The test group may be Autism Genetic Resource Exchange sample, as described in the Examples.

It is also possible to measure both specificity and sensitivity as follows:

Specificity: True negatives/True negatives+False positives
Sensitivity: True positives/True positives+True negatives

The specificity of the SNP set may be at least 70%, at least 80%, at least 85% or at least 90%.

The sensitivity of the SNP set may be at least 70%, at least 80%, at least 85% or at least 90%.

### SNP Investigation

The term “investigation” is used to mean that the presence or absence of a SNP in a given genome is determined.

Applicable diagnostic techniques include, but are not limited to, DNA sequencing including mini-sequencing, primer extension, hybridization with allele-specific oligonucleotides (ASO), oligonucleotide ligation assays (OLA), PCR using allele-specific primers (ARMS), dot blot analysis, flap probe cleavage approaches, restriction fragment length polymorphism (RFLP), kinetic PCR, and PCR-SSCP, fluorescent in situ hybridisation (FISH), pulsed field gel electrophoresis (PFGE) analysis, Southern blot analysis, single stranded conformation analysis (SSCA), denaturing gradient gel electrophoresis (DGGE), temperature gradient gel electrophoresis (TGGE), denaturing HPLC (DHPLC), and RNAse protection assays, all of which are known to the person skilled in the art.

For a known SNP, direct determination of the respective genotype is usually the method of choice. State of the art approaches for industrial high-throughput genotyping today rely on one of four different mechanisms: allele-specific primer extension, allele-specific hybridization, allele-specific oligonucleotide ligation and allele-specific cleavage of a flap probe (K wok, Pharmacogenomics 1, 95 (2000)). Sequencing or mini-sequencing protocols are part of the primer extension methods, e.g. genomic DNA sequencing, either manual or by automated means. Minisequencing (primer extension) technology is based on determining the sequence at a specific base by allowing the elongation of a primer by one base directly at the variant site (Landegren et al., Genome Res. 8: 769-76 (1998)). Short sequence reactions coupled with an alternative detection method are the nature of real time pyrophosphate sequencing (Nyren et al., Science 281:363 (1998)).

Allele-specific hybridization protocols rely on probes detecting one or several of the alleles present at the SNP positions. Several techniques were developed for detection of a hybridization event. In the 5′ nuclease assay and in the molecular beacon assay, the hybridization probes are fluorescently labelled and probe binding is detected via changes in the behaviour of the fluorescent label (Livak, Genet. Anal. 14, 143 (1999); Tyagi et al., Nat. Biotechnol. 16, 49 (1998)). Hybridization events may occur in liquid phase or with either the probe or the target bound to a solid surface.

An array (microchip) typically consists of thousands of distinct nucleotide probes which are built up in an array on a silicon chip. Nucleic acid to be analyzed is fluorescently labelled, and hybridized to the probes on the chip. This method is one of parallel processing of thousands of probes at once and can tremendously accelerate the analysis. In several publications the use of this method is described (Hacia et al., Nature Genetics 14, 441 (1996); Shoemaker et al., Nature Genetics 14, 450 (1996); Chee et al., Science 274, 610 (1996); DeRisi et al., Nature Genetics 14, 457 (1996), Fan et al., Genome Res, 10, 853 (2000)).

Allele-specific oligonucleotide ligation assays have a high specificity. Oligonucleotides differing in the allele-specific base at the 5′- or 3′-end are only processed in a ligation reaction if they are perfectly bound to the template at the respective oligonucleotide end. This method has been coupled with fluorescence resonance energy transfer (FRET) labeling to create a homogeneous assay system (Chen et al. Genome Res. 8, 549 (1998)). Allele-specific cleavage of a flap probe use the property of recently discovered flap endonucleases (cleavases) to cleave structures created by two overlapping oligonucleotides. In this approach two overlapping oligonucleotides are bound to the polymorphic site. That oligo which has had a perfect match to the target sequence is then detected by the cleavage reaction (Lyamichev et al., Nat. Biotechnol. 17:292 (1999)). Other methods which detect specific base variations usually allow only a lower throughput, such as the allele-specific oligonucleotide (ASO) hybridization. For allele-specific PCR, primers are used which hybridize at their 3′ ends to the target sequence. Only for alleles which are present, a respective PCR product is generated (Ruano and Kidd, Nucleic Acids Res 17, 8392 (1989)). A specificity increasing modification of allele-specific PCR is the Amplification Refractory Mutation System, as disclosed in European Patent Application Publication No. 0332435 and in Newton et al., Nucleic Acids Res 17, 2503 (1989). If the variations lead to changes in the specific recognition sites of nucleic acid processing, enzymes methodologies such as restriction fragment length polymorphism (RFLP) probes or PCR-RFLP methods may also be used to detect these variations.

Detection of SNPs may be accomplished by amplification, for instance by PCR, from genomic or cDNA and sequencing of the amplified nucleic acid or by molecular cloning of the relevant allele and sequencing the allele using techniques well known in the art.

### Kit

The present invention also provides kit for diagnosing an autism spectrum disorder (ASD), or predisposition to develop an ASD, in a subject, which comprises a plurality of primer pairs or probes capable of investigating a set of single nucleotide polymorphisms (SNPs) in a sample from the subject, wherein the set of SNPs is as defined above.

The kit may comprise a plurality of probes, each capable of hybridising specifically to one of the alternative forms of the SNP.

As used herein, the term “probe” refers to a nucleic acid (eg. an oligonucleotide or a polynucleotide sequence) that is complementary to a nucleic acid sequence present in a sample, such that the probe will specifically hybridize to the nucleic acid sequence present in the sample under appropriate conditions.

The kit may also comprise means for detecting the presence of a plurality of hybridization products, corresponding to each probe/SNP combination.

The probes may be gene probes, for example oligomeric DNA sequences of 15 to 50 bases which are synthesized with a variant base, to detect the presence of a SNP, or no variant bases, to detect the absence of a SNP.

The probe is then hybridized to the genome under stringent conditions allowing single base variant discrimination.

Alternatively the kit may comprise a plurality of primer pairs, using which each SNP may detected by:

a) amplifying the potential SNP-containing parts of the nucleic acid in said sample,
b) sequencing, e.g. mini-sequencing, the amplified nucleic acids; and
c) detecting the presence or absence of the SNPs in said sample.

The term “primer” as used herein refers to an oligonucleotide which is capable of acting as a point of initiation of synthesis when placed under conditions in which synthesis of a primer extension product which is complementary to a nucleic acid strand is induced, i.e. in the presence of nucleotides and an inducing agent—such as DNA polymerase and at a suitable temperature and pH.

The primers and/or probes may be labelled in order to facilitate their detection. Such labels (also known as reporters) include, but are not limited to, radioactive isotopes, fluorophores, chemiluminescent moieties, enzymes, enzyme substrates, enzyme cofactors, enzyme inhibitors, dyes, metal ions, metal sols, other suitable detectable markers—such as biotin or haptens and the like. Particular example of labels which may be used include, but are not limited to, fluorescein, 5(6)-carboxyfluorescein, Cyanine 3 (Cy3), Cyanine 5 (Cy5), rhodamine, dansyl, umbelliferone, Texas red, luminal, NADPH and horseradish peroxidase.

The probes and/or primers used in the kit hybridise specifically to their target nucleic acid sequence. They may, for example, hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature.

As used herein, high stringency refers to conditions that permit hybridisation of only those nucleic acid sequences that form stable hybrids in 1M Na+ at 65-68° C. High stringency conditions can be provided, for example, by hybridisation in an aqueous solution containing 6×SSC, 5×Denhardt's, 1% SDS (sodium dodecyl sulphate), 0.1 Na+ pyrophosphate and 0.1 mg/ml denatured salmon sperm DNA as non specific competitor.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

### Sample

The sample may be or may be derived from a biological sample, such as a blood sample, cheek swab, a biopsy specimen, a tissue extract, an organ culture or any other tissue or cell preparation from a subject.

In theory, the presence of SNP can be determined by extracting DNA from any tissue of the body.

The sample may be or may be derived from an ex vivo sample.

The sample may be or may be derived from whole blood or a fraction of whole blood.

Suitably, the sample is nucleic acid, such as genomic DNA.

### Subject

The subject may be a human. The subject may be a child under 10 years of age. The subject may be a child whose age or mental age is too low for reliable ASD assessment using behavioural tests. For example, the subject may be a child under 18 months of age. Additionally the subject may be an adolescent or adult.

The subject may be pre-implantation or post-implantation foetus.

Foetal cells for analysis can be obtained by amniocentesis, chorionic villus sampling (CVS), or drawing blood from the foetal umbilical cord, using methods known in the art. Pre-natal testing allows the likelihood of a subject to develop an ASD to be determined before birth, so this information can be taken into consideration throughout the child's babyhood and infancy.

Pre-implantation screening may be carried out, for example, during IVF procedures. Genetic material for analysis may be obtained, for example, from polar bodies using known techniques.

The subject may show some symptoms of an ASD. The subject may have been previously characterised as having an ASD by behavioural tests. Where the results of behavioural tests are ambiguous or inconclusive, the method of the present invention may be used to confirm the diagnosis.

The subject may have a family history of ASD.

### Solid Support

In the kit of the present invention, nucleic acid probes may be associated with a support or substrate to provide an array of nucleic acid probes to be used in an array assay. Suitably, the probe is pre-synthesized or obtained commercially, and then attached to the substrate or synthesized on the substrate, i.e., synthesized in situ on the substrate.

A specific method of nucleic acid hybridization that can be utilized is nucleic acid chip/array hybridization in which nucleic acids are present on a immobilized surface—such as a microarray and are subjected to hybridization techniques sensitive enough to detect minor changes in sequences.

As used herein, an “array” includes any two-dimensional or substantially two-dimensional (as well as a three-dimensional) arrangement of addressable regions bearing a particular chemical moiety or moieties (e.g., biopolymers—such as polynucleotide or oligonucleotide sequences (nucleic acids), polypeptides (e.g., proteins), carbohydrates, lipids, etc.). The array may be an array of polymeric binding agents—such as polypeptides, proteins, nucleic acids, polysaccharides or synthetic mimetics. Typically, the array is an array of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Array technology and the various techniques and applications associated with it is described generally in numerous textbooks and documents. These include Lemieux et al., 1998, Molecular Breeding 4, 277-289, Schena and Davis. Parallel Analysis with Biological Chips. in PCR Methods Manual (eds. M. Innis, D. Gelfand, J. Sninsky), Schena and Davis, 1999, Genes, Genomes and Chips. In DNA Microarrays: A Practical Approach (ed. M. Schena), Oxford University Press, Oxford, UK, 1999), The Chipping Forecast (Nature Genetics special issue; January 1999 Supplement), Mark Schena (Ed.), Microarray Biochip Technology, (Eaton Publishing Company), Cortes, 2000, The Scientist 14[17]:25, Gwynne and Page, Microarray analysis: the next revolution in molecular biology, Science, 1999 Aug. 6; and Eakins and Chu, 1999, Trends in Biotechnology, 17, 217-218.

Array technology overcomes the disadvantages with traditional methods in molecular biology, which generally work on a “one gene in one experiment” basis, resulting in low throughput and the inability to appreciate the “whole picture” of gene function. Array technology may be used in the context of the present invention to identify the presence or absence of some or all of the SNPs from the SNp set in the sample from the subject.

The SNP detection system (e.g. probes) may be fixed or immobilised onto a solid phase, preferably a solid substrate, to limit diffusion and admixing of the samples. Probes may be immobilised to a substantially planar solid phase, including membranes and non-porous substrates such as plastic and glass. Furthermore, the probes may be arranged in such a way that indexing (i.e., reference or access to a particular SNP) is facilitated. Typically the probes are applied as spots in a grid formation. Common assay systems may be adapted for this purpose. For example, an array may be immobilised on the surface of a microplate, either with multiple probes in a well, or with a single probe in each well. Furthermore, the solid substrate may be a membrane, such as a nitrocellulose or nylon membrane (for example, membranes used in blotting experiments). Alternative substrates include glass, or silica based substrates. Thus, the probes are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the probes on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14[11]:26. The number of distinct nucleic acid sequences, and hence spots or similar structures (i.e., array features), present on the array may vary, but is generally at least 2, usually at least 5 and more usually at least 10, where the number of different spots on the array may be as a high as 50, 100, 500, 1000, 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm².

Techniques for producing immobilised libraries of DNA molecules have been described in the art. Generally, most prior art methods described how to synthesise single-stranded nucleic acid molecule libraries, using for example masking techniques to build up various permutations of sequences at the various discrete positions on the solid substrate. U.S. Pat. No. 5,837,832, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

The array will include a plurality of different probes of different sequence covalently or non-covalently attached to, different and known locations on the substrate surface. The array may comprise a probe for each SNP in the SNP set.

To aid detection, targets and probes may be labelled with any readily detectable reporter, for example, a fluorescent, bioluminescent, phosphorescent, radioactive, etc reporter. Such reporters, their detection, coupling to targets/probes, etc are discussed elsewhere in this document. Labelling of probes and targets is also disclosed in Shalon et al., 1996, Genome Res 6(7):639-45

Specific examples of DNA arrays are as follow:

Format I: probe cDNA (5005,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 1999, 17, 217-218).

Format II: an array of oligonucleotide (20-25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such a DNA chip is sold by Affymetrix, Inc., under the GeneChip® trademark.

Data analysis is also an important part of an experiment involving arrays. The raw data from a microarray experiment typically are images, which need to be transformed into gene expression matrices—tables where rows represent for example genes, columns represent for example various samples such as tissues or experimental conditions, and numbers in each cell for example characterize the expression level of the particular gene in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J (2000) FEBS Lett 480(1):17-24.

SNPs may be detected using the BeadXpress Reader System (Illumina Inc., North America). See for example, U.S. Pat. No. 6,355,431. This system is a high-throughput, dual-colour laser detection system that enables scanning of a broad range of multiplexed assays developed using the VeraCode digital microbead technology. Unique VeraCode microbeads are scanned for their code and fluorescent signals, generating highly robust data quickly and efficiently. Downstream analysis is conducted using Illumina's BeadStudio data analysis software or other third-party analysis programs.

The invention will now be further described by way of Examples, which are meant to serve to assist one of ordinary skill in the art in carrying out the invention and are not intended in any way to limit the scope of the invention.

### EXAMPLES

### Example 1

### SVM Analysis of SNPs in Sample Comprising ASD Affected and Unaffected Individuals

Analysis of all individuals in the AGRE sample (see Materials and Methods), ‘Affected’ and ‘Unaffected’, using the ‘leave one out’ method, resulted in an overall accuracy of 87.6%, i.e. the algorithm predicted the correct diagnostic class for 87.6% of the total sample. The percentage accuracy was higher in affected individuals than in unaffected individuals (Table 1).

TABLE 1
Overall classification of all AGRE samples.

| | Total Sample (n) | Predicted (n) | Accuracy (%) |
|---|---|---|---|
| ASD Affected | 1385 | 1264 | 91.3 |
| Unaffected | 1494 | 1253 | 83.9 |
| Total | 2879 | 2517 | 87.6 |

Total sample: the total number of individuals in each diagnostic class.
Predicted: the total number correctly predicted by the SVM algorithm.
Accuracy: the total percentage accuracy achieved.

Using the following formulas, both specificity and sensitivity were measured:

### Specificity: True Negatives/True Negatives+False Positives=84%

### Sensitivity: True Positives/True Positives+True Negatives=92%

Of the 121 ASD individuals who were misclassified, 42 were twins (41 MZ & 1 DZ) and an additional 4 individuals were quadruplets. 23 of the individuals had a diagnosis of broader autism. 2 of the individuals misclassified had an original diagnosis of autism, however AGRE did not confirm these diagnoses upon their own assessments.

This supports the key assertions behind the invention that (i) clinicians make mistakes using standard diagnostic procedures; and (ii) genetic testing may be used to detect and correct these errors.

### Example 2

### Analysis with a Reduced Number of “Influential” SNPs

The whole genome sample described in Example 1 used a total of 390671 SNPs to achieve an overall accuracy of 87.6%. Subsequent analysis involved identifying, from the initial analysis, which were most influential to the classification, and repeating the analysis with a reduced number of “influential” (i.e. more highly weighted) SNPs.

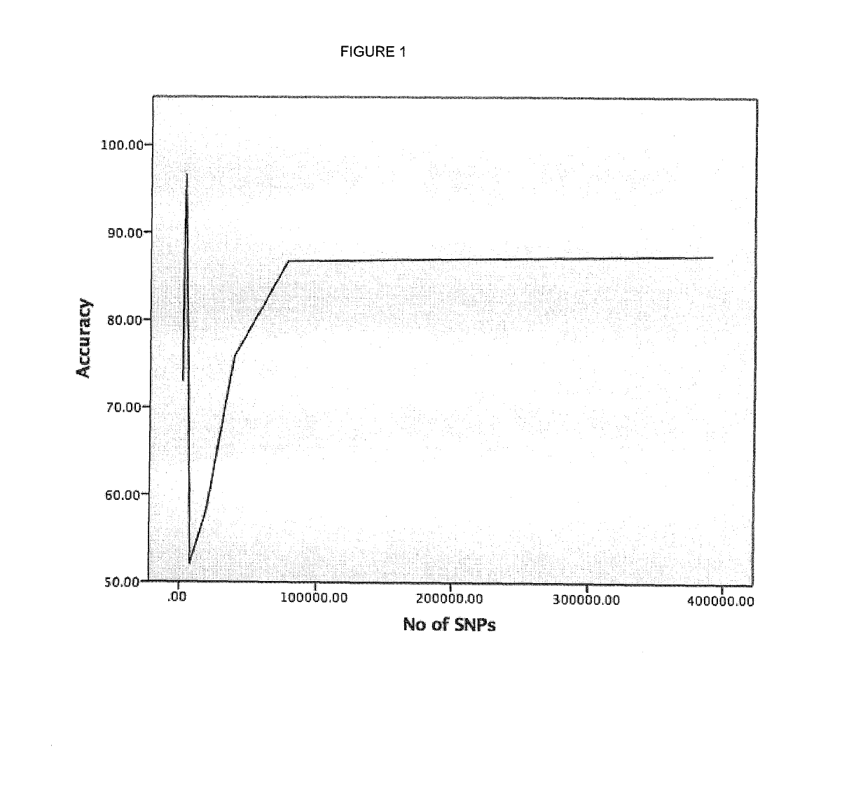

The results are shown in FIG. 1 and Table 2.

TABLE 2

| No of SNPs | Accuracy % |
|---|---|
| 390671 | 87.43 |
| 78134 | 86.73 |
| 39067 | 75.82 |
| 19534 | 58.18 |
| 7813 | 52.07 |
| 3907 | 89.09 |
| 3126 | 96.67 |
| 2345 | 89.00 |
| 1564 | 73.00 |

As shown in FIG. 1 and Table 2, maximal accuracy of over 96.6% is achieved with a SNP set of about 3126 of the most highly weighted SNPs from the whole genome sample. These SNPs, together with their respective weights, are shown in Table 3.

Increasing or decreasing the number of SNPs lowers the accuracy of the test, but SNP sets containing between 2345 and 3907 SNPs still result in an accuracy of at least 89%.

### Materials and Methods

### Sample

The Autism Genetic Resource Exchange (AGRE) sample was used, comprising of 1385 individuals with ASD and 1494 unaffected individuals. A total of 720 families were analysed, with at least one child diagnosed with autism using the ADI-R. The second (and subsequent) affected child had an AGRE classification of autism, broad spectrum (including Asperger's Syndrome and PDD-NOS) or Not Quite Autism (NQA, individuals who are no more than one point away from meeting autism criteria on any or all of the diagnostic domains). Ethnicity and race was self-reported at 69% white, 12% Hispanic/Latino, 10% Unknown, 5% mixed, 2.5% each Asian and African American, less than 1% Native Hawaiian/Pacific Islander and American Indian/Native Alaskan.

Within the total sample, one set of quadruplets', all diagnosed with autism were evident. Six sets of triplets (14 Autism/1 NQA/3 Unaffected), 43 dizygotic and 32 monozygotic twin pairs were noted (1 additional twin pair set, of unknown zygosity was also sampled). Of the individual twins, diagnosis was as follows—123 autism, 12 broad spectrum, 6 NQA, 4 unknown and 7 unaffected. Overall the diagnostic break down of all ASD individuals comprised of 87% autism, 8% broad spectrum and 5% NQA.

Genotyping was conducted using Affymetrix 5.0 chips at the Genetic Analysis Platform of the Broad Institute; full methods are described in (Weiss, L. A., Y. Shen, et al. (2008). N Engl J Med 358(7): 667-75).

Data was downloaded from www.agre.org, comprising of three files
1) .bed—file comprising all generated genotype data
2) .bim—map of all genotyped markers
3) .fam—family structures of all individuals genotyped
   From the .bed file, all SNPs found on the X and Y chromosomes were removed. Due to the high ratio of males to females diagnosed with ASD, it was though this in itself may be influential in the classification. The .bed file was divided into 2879 files, containing genotypic data for one individual.

### Support Vector Machine

A SVM analysis, using a linear kernel, was applied to the data using a leave-one-out procedure. In this procedure a single individual is withheld from the SVM training and then tested to assess whether they are affected or unaffected. The leave-one-out procedure was subsequently repeated 2879 times (for each individual) and the results averaged.

TABLE 3

| SNP | Weight |
|---|---|
| rs626479 | −0.0172 |
| rs6680471 | −0.0179 |
| rs6665853 | 0.0136 |
| rs4654500 | −0.0002 |
| rs4654511 | 0.0146 |
| rs12078298 | 0.0029 |
| rs2411738 | −0.0024 |
| rs12049256 | 0.0126 |
| rs9439603 | 0.0332 |
| rs2312464 | 0.007 |
| rs17030082 | −0.0119 |
| rs6679134 | 0.0119 |
| rs2506902 | 0.0056 |
| rs1292657 | 0.0323 |
| rs2487647 | 0.0312 |
| rs2744692 | 0.0198 |
| rs6694657 | −0.0023 |
| rs10927602 | 0.0225 |
| rs6693417 | 0.0224 |
| rs17471689 | 0.0345 |
| rs1934057 | 0.0119 |
| rs732725 | −0.0274 |
| rs2744749 | 0.0178 |
| rs11249045 | −0.0059 |
| rs10794668 | 0.0123 |
| rs430022 | 0.0113 |
| rs311480 | −0.0118 |
| rs566421 | −0.0161 |
| rs4908393 | 0.0105 |
| rs4654352 | 0.0103 |
| rs7547083 | −0.022 |
| rs6426343 | −0.0066 |
| rs10753264 | 0.0107 |
| rs669216 | 0.0206 |
| rs2796209 | −0.0063 |
| rs9659735 | −0.0331 |
| rs6696621 | −0.0031 |
| rs2180133 | −0.0341 |
| rs7517274 | −0.0307 |
| rs16825353 | −0.0078 |
| rs10493109 | 0.0106 |
| rs1034268 | 0.0056 |
| rs7518522 | −0.0288 |
| rs7365614 | 0.0267 |
| rs264025 | 0.0081 |
| rs945179 | 0.0239 |
| rs3766212 | 0.0178 |
| rs1502908 | −0.0032 |
| rs1967757 | −0.0007 |
| rs835342 | −0.0172 |
| rs1288332 | 0.0108 |
| rs1288587 | −0.0191 |
| rs953625 | 0.0054 |
| rs6683597 | −0.0282 |
| rs11206407 | 0.0034 |
| rs689258 | −0.014 |
| rs570218 | 0.0178 |
| rs498831 | 0.0165 |
| rs1749859 | −0.0087 |
| rs3815226 | −0.0023 |
| rs590621 | 0.0023 |
| rs13374752 | 0.0281 |
| rs3992634 | 0.0197 |
| rs1331855 | 0.0264 |
| rs706370 | −0.0104 |
| rs1557061 | 0.0153 |
| rs3005896 | 0.0012 |
| rs12138574 | 0.0033 |
| rs4477326 | 0.0222 |
| rs4912230 | 0.0236 |
| rs1524707 | 0.0257 |
| rs4244011 | −0.0018 |
| rs2760484 | 0.0325 |
| rs6679571 | 0.0214 |
| rs585368 | −0.02 |
| rs11207575 | 0.0167 |
| SNP_A- | −0.0021 |
| 4277480 | |
| rs11207831 | −0.0086 |
| rs10889383 | −0.0088 |
| rs6662848 | −0.004 |
| rs2179811 | −0.0227 |
| rs6588064 | 0.0146 |
| rs825191 | −0.0152 |
| rs2186122 | −0.0038 |
| rs7551528 | 0.0004 |
| rs7516251 | 0.0142 |
| rs2815351 | −0.0063 |
| rs17375018 | 0.0327 |
| rs12083789 | −0.032 |
| rs288929 | 0.0061 |
| rs617344 | 0.0092 |
| rs11209455 | 0.0106 |
| rs10736412 | 0.0198 |
| rs11162095 | 0.0343 |
| rs9970671 | −0.0282 |
| rs4949764 | −0.0111 |
| rs10493649 | −0.0022 |
| rs17105569 | 0.0247 |
| rs10874241 | −0.0312 |
| rs7542573 | −0.0193 |
| rs12057556 | −0.0203 |
| rs772604 | 0.0247 |
| rs10782601 | −0.0071 |
| rs6662386 | −0.0021 |
| rs1188909 | −0.0228 |
| rs4414050 | 0.0123 |
| rs2249591 | −0.031 |
| rs12565150 | −0.0068 |
| rs12132107 | 0.0028 |
| rs2064662 | 0.0043 |
| rs6693882 | 0.0195 |
| rs1889060 | −0.0212 |
| rs12757095 | −0.0299 |
| rs387176 | 0.0194 |
| rs396954 | 0.0198 |
| rs12137571 | −0.0226 |
| rs2336015 | 0.0066 |
| rs11577194 | 0.0076 |
| rs17631306 | 0.0158 |
| rs3393 | 0.0183 |
| SNP_A- | 0.0231 |
| 1962128 | |
| rs11102368 | −0.0063 |
| rs4839202 | −0.0054 |
| rs11102374 | −0.0197 |
| rs2998359 | −0.0262 |
| rs4838994 | 0.0277 |
| SNP_A- | 0.0168 |
| 4240881 | |
| rs12145661 | −0.009 |
| rs568359 | 0.0102 |
| rs4659126 | 0.0069 |
| rs1325939 | 0.0231 |
| rs2275236 | −0.0252 |
| rs10494273 | 0.0233 |
| rs6587671 | −0.0089 |
| rs1923496 | −0.0158 |
| rs2146116 | −0.0039 |
| rs10908448 | −0.0233 |
| rs954916 | −0.0101 |
| rs9427242 | 0.0002 |
| rs822430 | 0.0088 |
| rs4661138 | 0.0121 |
| rs11265186 | −0.0272 |
| rs2501348 | 0.0104 |
| rs4596880 | −0.0061 |
| rs2490438 | 0.0084 |
| rs10919117 | −0.0135 |
| rs2661810 | −0.0058 |
| rs11580624 | −0.0167 |
| rs10753642 | 0.0012 |
| rs1494409 | 0.0172 |
| rs1494408 | −0.0016 |
| rs6675438 | −0.0205 |
| rs1343546 | 0.0138 |
| rs2027573 | 0.019 |
| rs6697712 | −0.0012 |
| rs6426958 | 0.0071 |
| rs1532482 | 0.0145 |
| rs869714 | 0.0057 |
| rs885458 | −0.011 |
| rs2143312 | 0.0074 |
| rs10918931 | −0.007 |
| rs16863926 | −0.0196 |
| rs2224396 | 0.0179 |
| rs12075807 | 0.0025 |
| rs484686 | −0.0337 |
| rs912300 | 0.0264 |
| rs6694387 | 0.0145 |
| rs9425287 | −0.0049 |
| rs6689901 | 0.0121 |
| rs10732999 | 0.0205 |
| rs10913355 | 0.0273 |
| rs12033565 | −0.0189 |
| rs7522303 | −0.001 |
| rs2811280 | −0.0054 |
| rs1570807 | 0.0144 |
| rs3845427 | 0.0159 |
| rs477956 | −0.0165 |
| rs6703122 | 0.0068 |
| rs6692352 | −0.005 |
| rs6704003 | −0.012 |
| rs10911583 | 0.0191 |
| rs6424983 | −0.0263 |
| rs10801058 | 0.0304 |
| rs1568133 | 0.0114 |
| rs16836373 | −0.0067 |
| rs634727 | −0.018 |
| rs10733086 | −0.0013 |
| rs1332660 | 0.0176 |
| rs2224873 | −0.0369 |
| rs2359372 | 0.0093 |
| rs3767735 | 0.0352 |
| rs1892432 | 0.003 |
| rs2249156 | −0.0006 |
| rs2248967 | −0.0006 |
| rs1572789 | −0.0126 |
| rs6700264 | 0.0191 |
| rs2486933 | 0.0146 |
| rs2486942 | −0.0074 |
| rs4543864 | 0.0173 |
| rs1997034 | 0.0178 |
| rs2282450 | −0.0065 |
| rs12407361 | −0.0078 |
| rs11579772 | −0.0008 |
| rs7552993 | 0.0033 |
| rs684431 | 0.0107 |
| rs1933573 | −0.0169 |
| rs3753522 | 0.0057 |
| rs1962735 | 0.0239 |
| rs2294850 | −0.0073 |
| rs926579 | 0.0037 |
| rs10863900 | −0.0007 |
| rs4951534 | 0.0229 |
| rs1509866 | 0.0154 |
| rs11117658 | 0.0307 |
| rs11117796 | −0.0148 |
| rs1602269 | −0.0023 |
| rs2798631 | 0.0174 |
| rs10863404 | −0.0094 |
| rs4428898 | −0.023 |
| rs6694088 | 0.0098 |
| rs2210977 | −0.0024 |
| rs2970199 | −0.0255 |
| rs2790762 | 0.0019 |
| rs6679430 | 0.0233 |
| rs6676201 | −0.0041 |
| rs710824 | −0.0151 |
| rs10916464 | −0.0109 |
| rs7538437 | −0.014 |
| rs1160075 | −0.0268 |
| rs867844 | −0.0214 |
| rs6698696 | 0.0051 |
| rs549209 | −0.0044 |
| rs6659304 | 0.0055 |
| rs4659838 | −0.022 |
| rs291356 | −0.0072 |
| rs2853599 | −0.01 |
| rs2385494 | −0.0077 |
| rs1266164 | 0.006 |
| rs1252252 | 0.0001 |
| rs4659743 | 0.0021 |
| rs10802581 | −0.0097 |
| rs4659491 | −0.009 |
| rs16835116 | 0.011 |
| rs7525233 | −0.0271 |
| rs6657299 | −0.0227 |
| rs1984165 | 0.007 |
| rs10926147 | −0.0228 |
| rs1539098 | 0.0146 |
| rs3765814 | −0.0013 |
| rs6669036 | 0.0049 |
| rs6696527 | −0.0018 |
| rs2809979 | −0.0081 |
| rs10926897 | 0.0217 |
| rs544739 | −0.0016 |
| rs2786694 | −0.0119 |
| rs1093961 | 0.0034 |
| rs1069217 | 0.0056 |
| rs10924359 | −0.0218 |
| rs3124124 | 0.0171 |
| rs6671004 | 0.0129 |
| rs2386548 | −0.0136 |
| rs1144812 | 0.0244 |
| rs4382761 | −0.0093 |
| rs1364648 | 0.0155 |
| rs1451196 | 0.0086 |
| rs6738683 | 0.0168 |
| rs6548128 | −0.0314 |
| rs10174005 | 0.0115 |
| rs792105 | −0.0052 |
| rs7604344 | −0.0249 |
| rs1381514 | 0.0102 |
| rs1461312 | −0.0205 |
| rs13382991 | −0.001 |
| rs10167072 | −0.0093 |
| rs17681545 | 0.0194 |
| rs7572345 | 0.0197 |
| rs328634 | −0.0259 |
| rs1003187 | −0.0094 |
| rs4668666 | −0.0266 |
| rs6432085 | −0.0011 |
| rs16856887 | 0.0039 |
| rs7594176 | −0.0297 |
| rs10167277 | −0.0033 |
| rs1225214 | 0.0352 |
| rs6432270 | 0.0071 |
| rs17344070 | −0.0206 |
| rs2571642 | −0.0347 |
| rs10189450 | 0.0221 |
| rs10803676 | 0.0235 |
| rs13385499 | −0.0305 |
| rs12714264 | 0.0071 |
| rs312970 | 0.0016 |
| rs576203 | 0.0065 |
| rs7570872 | −0.0207 |
| rs11127215 | −0.0142 |
| rs207413 | −0.0111 |
| rs212708 | 0.0102 |
| rs1534350 | 0.0018 |
| rs4670550 | 0.0013 |
| rs1015696 | −0.0171 |
| rs7588747 | 0.0251 |
| rs173023 | −0.0186 |
| rs13010748 | 0.0096 |
| rs297150 | −0.0056 |
| rs7582507 | −0.0215 |
| rs6746891 | 0.0119 |
| rs11688286 | 0.0207 |
| rs1517024 | 0.0361 |
| rs4082957 | −0.0028 |
| rs17033378 | 0.0165 |
| rs11678872 | 0.016 |
| rs12712996 | 0.0064 |
| rs1574380 | 0.0048 |
| rs6738387 | −0.0048 |
| rs2300443 | −0.0124 |
| rs13000157 | −0.0153 |
| rs7355586 | −0.0374 |
| rs17489439 | −0.0243 |
| rs1159982 | 0.006 |
| rs12328023 | 0.0287 |
| rs7557799 | 0.0276 |
| rs2033411 | −0.0211 |
| rs4672103 | −0.0027 |
| rs11673760 | −0.0195 |
| rs820985 | 0.0133 |
| rs759250 | 0.0345 |
| rs11691214 | −0.0023 |
| rs4671398 | −0.0061 |
| rs1011066 | −0.0029 |
| rs17692393 | −0.0008 |
| rs2540959 | −0.0126 |
| rs1420183 | −0.0284 |
| rs11678288 | 0.0036 |
| rs719832 | −0.0111 |
| rs6743599 | −0.0051 |
| rs3771527 | 0.0125 |
| rs12617676 | −0.0058 |
| rs4852430 | −0.0143 |
| rs4510249 | 0.0007 |
| rs1455385 | 0.0065 |
| rs6738834 | −0.0101 |
| rs17022288 | 0.0106 |
| rs6749875 | 0.0355 |
| rs11689667 | −0.0103 |
| rs883650 | −0.0062 |
| rs9677221 | −0.0079 |
| rs6738956 | −0.0114 |
| rs9309628 | −0.0345 |
| rs17704088 | −0.0072 |
| rs6713310 | 0.0046 |
| rs921423 | 0.0167 |
| rs1010387 | 0.0322 |
| rs4632400 | 0.0056 |
| rs17493655 | −0.0018 |
| rs11686712 | −0.0126 |
| rs11683001 | −0.0431 |
| rs4603782 | 0.0026 |
| rs12712130 | 0.0053 |
| rs1861228 | −0.0229 |
| rs12998183 | −0.015 |
| rs6543426 | 0.0031 |
| rs1524297 | 0.0081 |
| rs1524287 | 0.0079 |
| rs4676274 | −0.0125 |
| rs6754115 | −0.0191 |
| rs884448 | 0.0104 |
| rs10181720 | 0.0236 |
| rs17047362 | −0.0024 |
| rs6737733 | 0.004 |
| rs1433526 | −0.0105 |
| rs17584619 | −0.0147 |
| rs10207133 | −0.0125 |
| rs11679589 | 0.0068 |
| rs718867 | 0.017 |
| rs297479 | −0.017 |
| rs299566 | −0.0109 |
| rs13002629 | −0.0133 |
| rs12468639 | −0.0112 |
| rs11123081 | −0.0233 |
| rs13390374 | 0.0356 |
| rs831360 | −0.0116 |
| rs3820757 | 0.0187 |
| rs4150477 | 0.009 |
| rs840875 | −0.0212 |
| rs7578253 | 0.0087 |
| rs7586789 | 0.0169 |
| rs6431151 | 0.0241 |
| rs6749712 | 0.0011 |
| rs6727803 | −0.0171 |
| rs1446749 | 0.0114 |
| rs16831992 | 0.0075 |
| rs936835 | −0.0145 |
| rs443719 | 0.0337 |
| rs10197153 | −0.0294 |
| rs351673 | 0.0042 |
| rs9287378 | 0.0115 |
| rs13421583 | −0.0263 |
| rs6430136 | 0.0004 |
| rs1356738 | 0.0334 |
| rs1519768 | 0.0137 |
| rs1519800 | −0.0112 |
| rs298247 | −0.0078 |
| rs16842681 | −0.0024 |
| rs12692654 | −0.0037 |
| rs1369252 | 0.0005 |
| rs2083482 | −0.0345 |
| rs4340537 | 0.0048 |
| rs6732793 | −0.0132 |
| rs1540821 | 0.0156 |
| rs700542 | 0.0235 |
| rs17251018 | 0.0007 |
| rs741378 | 0.019 |
| rs7590275 | −0.0045 |
| rs10206361 | 0.0189 |
| rs2194720 | 0.0091 |
| rs3754764 | 0.0147 |
| rs2113807 | 0.0271 |
| rs4893825 | 0.0445 |
| rs4893966 | 0.0232 |
| rs6751992 | −0.0034 |
| rs260059 | 0.0244 |
| rs10186570 | −0.0158 |
| rs12466031 | −0.0348 |
| rs2368384 | −0.0149 |
| rs11902682 | −0.0012 |
| rs826168 | −0.0311 |
| rs7560722 | 0.0057 |
| rs10166420 | 0.0297 |
| rs887701 | −0.0161 |
| rs1378156 | −0.0215 |
| rs4853575 | −0.0173 |
| rs7558504 | −0.0068 |
| rs10931635 | −0.0123 |
| rs1392658 | 0.0158 |
| rs11888904 | −0.0154 |
| rs7558972 | −0.0314 |
| rs12614240 | 0.0117 |
| rs295134 | 0.0197 |
| rs4233996 | 0.0039 |
| rs295149 | 0.0309 |
| rs1369841 | 0.0181 |
| rs11892551 | −0.0171 |
| rs6757529 | 0.0117 |
| rs10195536 | −0.0232 |
| rs3820901 | 0.0144 |
| rs16844213 | 0.0098 |
| rs3821136 | −0.022 |
| rs10167685 | 0.015 |
| rs2662683 | −0.0284 |
| rs4673821 | 0.0126 |
| rs12612329 | −0.0136 |
| rs828910 | 0.0079 |
| rs17779951 | 0.0155 |
| rs750764 | −0.0123 |
| rs17226763 | −0.0064 |
| rs6723377 | 0.0106 |
| rs6733393 | −0.0056 |
| rs1371551 | 0.0095 |
| rs3845841 | 0.0072 |
| rs1596395 | 0.0107 |
| rs9288601 | −0.0184 |
| rs10208046 | 0.0171 |
| rs6744449 | 0.0087 |
| rs3811514 | −0.0451 |
| rs6436790 | 0.0171 |
| rs3106301 | 0.0177 |
| rs6437020 | 0.0145 |
| rs13395911 | −0.0165 |
| rs689101 | −0.0064 |
| rs972513 | 0.0132 |
| rs2316434 | 0.0076 |
| rs7571980 | −0.0146 |
| rs2292708 | 0.0099 |
| rs10192532 | −0.0154 |
| rs2573712 | 0.0297 |
| rs10187736 | −0.0219 |
| rs821501 | −0.0279 |
| rs7608438 | −0.0093 |
| rs2648466 | 0.0306 |
| rs1827106 | −0.0078 |
| rs11128826 | −0.0376 |
| rs4434131 | −0.0194 |
| rs1143977 | −0.0028 |
| rs1032783 | 0.002 |
| rs1666325 | −0.0204 |
| rs17018141 | 0.004 |
| rs427502 | −0.0226 |
| rs17023520 | −0.0172 |
| rs4685803 | 0.0167 |
| rs3804992 | −0.0004 |
| rs7637793 | 0.0029 |
| rs11130339 | −0.023 |
| rs286588 | 0.0053 |
| rs10490889 | 0.0086 |
| rs2117788 | 0.0025 |
| rs5010733 | −0.0233 |
| rs571701 | −0.0125 |
| rs341795 | −0.0003 |
| rs420537 | −0.0238 |
| rs709165 | −0.0188 |
| rs299651 | −0.0022 |
| rs1618545 | 0.0136 |
| rs2633442 | 0.0035 |
| rs9831765 | 0.0225 |
| rs2881980 | 0.0149 |
| rs4684901 | 0.0128 |
| rs4684131 | 0.0147 |
| rs9865654 | 0.0164 |
| rs981694 | 0.0074 |
| rs1587378 | 0.0393 |
| rs10510530 | 0.023 |
| rs13317243 | 0.0072 |
| rs6550755 | −0.0193 |
| rs9863976 | −0.0219 |
| rs13072262 | 0.0182 |
| rs322680 | −0.0127 |
| rs922289 | 0.0186 |
| rs17026472 | −0.0023 |
| rs12630254 | −0.013 |
| rs6781673 | 0.0338 |
| rs9853831 | −0.0089 |
| rs25506 | 0.0244 |
| rs2197728 | 0.0074 |
| rs6799641 | 0.0074 |
| rs999745 | −0.0334 |
| rs6806372 | 0.0178 |
| rs2693477 | 0.0072 |
| rs7431934 | 0.0067 |
| rs416183 | −0.013 |
| rs7639483 | 0.0287 |
| rs9846284 | 0.0018 |
| rs2191031 | −0.0272 |
| rs13314659 | 0.0141 |
| rs4446245 | 0.0116 |
| rs17054250 | −0.0013 |
| SNP_A- | −0.0064 |
| 1880070 | |
| rs3864001 | −0.0033 |
| rs4083342 | −0.0065 |
| rs17054340 | −0.0062 |
| rs1461796 | 0.0288 |
| rs6445725 | 0.0154 |
| rs9683127 | 0.0113 |
| rs641035 | 0.0195 |
| rs4637258 | −0.0104 |
| rs11708862 | 0.005 |
| rs734184 | 0.0158 |
| rs2366968 | 0.033 |
| rs17258256 | −0.0314 |
| rs6785140 | −0.0136 |
| rs9834435 | −0.0221 |
| rs9840800 | 0.0292 |
| rs11918950 | −0.0173 |
| rs2600859 | −0.0137 |
| rs17066787 | 0.0193 |
| rs4254651 | −0.0189 |
| rs6445454 | 0.0033 |
| rs11705970 | 0.0079 |
| rs7627319 | −0.0332 |
| rs6772684 | 0.0151 |
| rs1288824 | 0.0041 |
| rs13087868 | −0.0045 |
| rs4677093 | 0.0065 |
| rs6549724 | −0.0039 |
| rs17745164 | 0.0231 |
| rs9852230 | 0.0188 |
| rs6548639 | −0.0086 |
| rs17749340 | −0.0005 |
| rs13327580 | 0.0184 |
| rs6795503 | 0.0003 |
| rs17018312 | −0.0336 |
| rs2372744 | 0.0014 |
| rs2639220 | 0.0101 |
| rs6548788 | 0.0261 |
| rs9859573 | −0.0115 |
| rs291961 | 0.0091 |
| rs188349 | 0.0062 |
| rs10511061 | 0.0284 |
| rs11127978 | −0.0104 |
| rs6767207 | −0.0136 |
| rs1355226 | −0.0038 |
| rs724972 | −0.0152 |
| rs7631619 | −0.0077 |
| rs6785834 | −0.0008 |
| rs13068356 | −0.0039 |
| rs1470574 | −0.002 |
| rs16849393 | −0.0023 |
| rs16830554 | 0.0138 |
| rs10804622 | 0.0374 |
| rs4928169 | −0.0151 |
| rs1318859 | 0.0119 |
| rs4928057 | 0.0062 |
| rs1507436 | −0.0099 |
| rs9822356 | −0.0057 |
| rs1241150 | 0.0035 |
| rs12488048 | 0.0102 |
| rs9824049 | 0.0097 |
| rs11927739 | 0.0234 |
| rs7643193 | 0.0055 |
| rs13100164 | 0.0079 |
| rs4484197 | −0.0085 |
| rs2715750 | −0.0207 |
| rs9815868 | 0.0066 |
| rs1462302 | −0.0075 |
| rs6805801 | −0.0063 |
| rs2971352 | −0.0145 |
| rs6784753 | 0.018 |
| rs11720141 | 0.0164 |
| rs1553191 | −0.0184 |
| rs9289082 | −0.0078 |
| rs3772132 | 0.0003 |
| rs1259494 | 0.0008 |
| rs7628094 | 0.0109 |
| rs2084399 | 0.0268 |
| rs11707393 | 0.0221 |
| rs2403313 | −0.0217 |
| rs11713445 | −0.0098 |
| rs995538 | −0.0175 |
| rs149862 | 0.0195 |
| rs16849309 | 0.0198 |
| rs7632817 | 0.0174 |
| rs4616638 | −0.0069 |
| rs4527375 | −0.0052 |
| rs9811430 | −0.0209 |
| rs9865339 | 0.0043 |
| rs1195268 | −0.0158 |
| rs1026828 | −0.0089 |
| rs1026826 | −0.0085 |
| rs2889217 | −0.0035 |
| rs6792834 | −0.0085 |
| rs11915439 | 0.0134 |
| rs10804716 | −0.0104 |
| rs1562788 | 0.0144 |
| rs1850963 | 0.0192 |
| rs4679958 | 0.0299 |
| rs161792 | 0.0081 |
| rs659379 | −0.0139 |
| rs701142 | 0.0245 |
| rs13080908 | −0.0203 |
| rs7615026 | −0.0046 |
| rs6440995 | 0.012 |
| rs344028 | −0.0153 |
| rs11914959 | −0.0154 |
| rs987724 | 0.0051 |
| rs9867766 | 0.0088 |
| rs11720579 | 0.0091 |
| rs10451916 | 0.0255 |
| rs1392799 | 0.0186 |
| rs9831830 | 0.0201 |
| rs11717115 | −0.0107 |
| rs6794184 | −0.0339 |
| rs1879803 | −0.0216 |
| rs1497762 | −0.0211 |
| rs206313 | 0.0083 |
| rs206310 | 0.0049 |
| rs3849450 | −0.0328 |
| rs11719240 | −0.004 |
| rs1407542 | −0.0246 |
| rs7628732 | −0.0017 |
| rs298755 | 0.0003 |
| rs10513619 | −0.0205 |
| rs1440684 | −0.0382 |
| rs9990094 | 0.0064 |
| rs10222648 | 0.0088 |
| rs12485932 | 0.0142 |
| rs6778086 | 0.0162 |
| rs7623033 | −0.0108 |
| rs4855084 | 0.0099 |
| rs17188166 | −0.0198 |
| rs2700856 | 0.0138 |
| rs1002767 | 0.0142 |
| rs4912577 | −0.0032 |
| rs11715222 | 0.0383 |
| rs6791954 | −0.0422 |
| rs12696555 | 0.0156 |
| rs9853541 | 0.0104 |
| rs974671 | 0.0233 |
| rs12330507 | 0.017 |
| rs16863396 | 0.0244 |
| rs13084980 | −0.0243 |
| rs9879356 | 0.0192 |
| rs9853187 | 0.008 |
| rs1875731 | 0.0137 |
| rs2708309 | −0.011 |
| rs2134634 | −0.011 |
| rs9854346 | 0.0113 |
| rs6785291 | 0.0236 |
| rs11185519 | 0.0013 |
| rs7649045 | −0.0229 |
| rs6600769 | 0.0174 |
| rs1014947 | −0.0121 |
| rs1419046 | 0.0007 |
| rs2251457 | 0.0032 |
| rs2471347 | 0.0134 |
| rs2968684 | −0.0403 |
| rs7693293 | −0.0044 |
| rs6838792 | 0.002 |
| rs750518 | 0.0006 |
| rs3821920 | −0.0204 |
| rs12505562 | 0.0393 |
| rs16837510 | 0.0124 |
| rs16837871 | 0.02 |
| rs6826497 | −0.0068 |
| rs7376617 | 0.0003 |
| rs7378331 | 0.0027 |
| rs6824720 | −0.0333 |
| rs10937714 | 0.0284 |
| rs755403 | 0.0279 |
| rs4569755 | −0.0231 |
| rs1344223 | −0.0083 |
| rs1048506 | −0.0289 |
| rs3775940 | −0.0338 |
| rs16877315 | 0.0262 |
| rs1558402 | 0.0237 |
| rs1563254 | −0.0033 |
| rs6448930 | 0.0221 |
| rs6448968 | −0.0146 |
| rs10489083 | −0.0091 |
| rs13113093 | −0.014 |
| rs3843422 | −0.0102 |
| rs12641991 | 0.0188 |
| rs7663994 | −0.0153 |
| rs13128332 | −0.02 |
| rs12503223 | 0.0364 |
| rs4698512 | 0.0139 |
| rs4698515 | 0 |
| rs604768 | −0.0101 |
| rs12507442 | 0.0262 |
| rs921371 | 0.0253 |
| rs12646671 | 0.0094 |
| rs3815414 | 0.0198 |
| rs1006722 | 0.0149 |
| rs877323 | −0.0119 |
| rs10489014 | −0.011 |
| rs169778 | −0.0089 |
| rs1024120 | 0.0179 |
| rs10517186 | 0.0019 |
| rs11930738 | −0.0057 |
| rs902658 | 0.0284 |
| rs874498 | 0.0261 |
| rs16990031 | 0.007 |
| SNP_A- | −0.0057 |
| 2123334 | |
| rs1158162 | 0.0262 |
| rs16990692 | −0.003 |
| rs4421010 | 0.0044 |
| rs7690708 | −0.0037 |
| rs6829898 | 0.0137 |
| rs11735595 | −0.0152 |
| rs2068783 | 0.0035 |
| rs7682470 | −0.0115 |
| rs10026397 | 0.0007 |
| rs10026359 | −0.0141 |
| rs7691499 | −0.0088 |
| rs4481267 | 0.0117 |
| rs17460101 | −0.0152 |
| rs12501005 | 0.0211 |
| rs7679812 | 0.0276 |
| rs6839710 | 0.0182 |
| rs2048545 | −0.0147 |
| rs16858583 | 0.0236 |
| rs4560384 | −0.0084 |
| rs12374260 | −0.0104 |
| rs1466998 | 0.0014 |
| rs11725773 | 0.0151 |
| rs17698672 | 0.0122 |
| rs7677726 | 0.0078 |
| rs4864471 | 0.0017 |
| rs11727673 | −0.0266 |
| rs2646333 | 0.025 |
| rs11726609 | −0.0042 |
| rs17727883 | −0.0008 |
| rs17827094 | −0.0297 |
| rs2570100 | 0.0098 |
| rs2611162 | 0.0167 |
| rs1522095 | 0.0083 |
| rs6554523 | 0.0189 |
| rs10001785 | 0.0228 |
| rs7689250 | −0.0196 |
| rs1551324 | −0.0096 |
| rs12501149 | −0.0003 |
| rs9312096 | −0.013 |
| rs7681297 | −0.017 |
| rs725761 | −0.0147 |
| rs11131312 | 0.0229 |
| rs9685679 | 0.0097 |
| rs1947275 | −0.0208 |
| rs7693819 | −0.0065 |
| rs12510678 | 0.0052 |
| rs2347747 | −0.0082 |
| rs10517943 | −0.0174 |
| rs4146477 | −0.0172 |
| rs17735539 | −0.0466 |
| rs1481261 | −0.0094 |
| rs10024794 | 0.0168 |
| rs2646295 | −0.0237 |
| rs13136907 | −0.0136 |
| rs2130648 | −0.0158 |
| rs732317 | 0.0169 |
| rs11734344 | −0.0103 |
| rs6816344 | −0.0093 |
| rs3113927 | 0.0068 |
| rs10022778 | −0.0056 |
| rs4859755 | −0.0039 |
| rs1477314 | −0.0207 |
| rs1559119 | −0.01 |
| rs6534293 | 0.0204 |
| rs6535252 | 0.031 |
| rs895087 | −0.0028 |
| rs530222 | −0.0032 |
| rs7683970 | 0.0046 |
| rs17355265 | −0.0347 |
| rs7694527 | 0.0066 |
| rs17009114 | 0.0018 |
| rs1373666 | 0.0009 |
| rs340196 | 0.0144 |
| rs1460767 | −0.0127 |
| rs1978672 | 0.0185 |
| rs10009760 | 0.0164 |
| rs1849809 | 0.0126 |
| rs7676210 | −0.0112 |
| rs17265801 | 0.0053 |
| rs10014892 | −0.0125 |
| rs6837448 | 0.0081 |
| rs6811974 | 0.0186 |
| rs10050093 | −0.0069 |
| rs6857138 | −0.0203 |
| rs6843996 | 0.0361 |
| rs10516420 | −0.0092 |
| rs4698964 | 0.0242 |
| rs233983 | −0.0145 |
| rs2189156 | 0.044 |
| rs11722542 | 0.0107 |
| rs4698784 | 0.0069 |
| rs4541508 | 0.0287 |
| rs4698797 | −0.0167 |
| rs12499868 | −0.0192 |
| rs17551888 | −0.0286 |
| rs10213287 | −0.0052 |
| rs6836139 | −0.0252 |
| rs11098223 | 0.0017 |
| rs10516630 | 0.0068 |
| rs11562835 | −0.0096 |
| rs6816919 | −0.014 |
| rs6847160 | −0.0203 |
| rs6823449 | 0.0234 |
| rs9993199 | −0.0329 |
| rs2389803 | −0.0285 |
| rs13138476 | 0.0095 |
| rs1963648 | 0.0106 |
| rs870254 | 0.0074 |
| rs1358229 | 0.01 |
| rs10026751 | 0.0249 |
| rs7690678 | −0.0326 |
| rs11099041 | 0.0171 |
| rs9917861 | 0.024 |
| rs12645403 | 0.0312 |
| rs17538880 | −0.0033 |
| rs11935540 | −0.0035 |
| rs17542701 | −0.0157 |
| rs976018 | −0.0121 |
| rs12639827 | 0.0001 |
| rs4541564 | 0.0118 |
| rs890444 | −0.0114 |
| SNP_A- | 0.0202 |
| 1958121 | |
| rs11726181 | −0.006 |
| rs2246759 | 0.0072 |
| rs6831959 | 0.0154 |
| rs2056383 | 0.0035 |
| rs1426888 | 0.009 |
| rs982076 | −0.0418 |
| rs1994624 | 0.0304 |
| rs10002297 | −0.0043 |
| rs360954 | 0.0153 |
| rs1425398 | 0.0113 |
| rs13143024 | 0.0218 |
| rs7696772 | 0.0168 |
| rs3796564 | 0.006 |
| rs11737459 | 0.0025 |
| rs12509506 | 0.0259 |
| rs10014567 | −0.0126 |
| rs12640506 | 0.0111 |
| rs4373123 | 0.0038 |
| rs13125269 | 0.0043 |
| rs17585174 | −0.036 |
| rs11726531 | −0.0151 |
| rs1388886 | −0.0041 |
| rs6854438 | −0.0086 |
| rs2712124 | 0.0284 |
| rs12511925 | −0.0263 |
| rs17054590 | 0.0157 |
| rs10866351 | 0.0213 |
| rs7377766 | 0.0091 |
| rs11132967 | −0.0315 |
| rs13141582 | 0.006 |
| rs4695953 | 0.0076 |
| rs4513542 | −0.0259 |
| rs4695963 | −0.01 |
| rs446082 | 0.005 |
| rs6848215 | 0.0148 |
| rs921310 | 0.0154 |
| rs6553973 | 0.0028 |
| rs10001364 | −0.0152 |
| rs2706013 | 0.0006 |
| rs4470674 | −0.0292 |
| rs11721398 | 0.0072 |
| rs1379988 | 0.0252 |
| rs922029 | −0.0064 |
| rs11131966 | −0.0146 |
| rs17279535 | −0.0442 |
| rs10015276 | 0.0008 |
| rs2100696 | −0.0029 |
| rs7661192 | −0.0223 |
| rs7693462 | 0.022 |
| rs11727792 | 0.0005 |
| rs961534 | −0.0161 |
| rs11722354 | 0.0063 |
| rs11132220 | −0.0214 |
| rs7349708 | 0.0072 |
| rs314102 | 0.0148 |
| rs2132668 | 0.0008 |
| rs10866321 | 0.0313 |
| rs4956990 | −0.0265 |
| rs12521051 | 0.0352 |
| rs2883374 | −0.0109 |
| rs13153232 | 0.008 |
| rs424339 | −0.0057 |
| rs380259 | −0.0146 |
| rs1609462 | −0.0036 |
| rs6555155 | −0.0165 |
| rs7727714 | 0.0238 |
| rs13165701 | 0.0214 |
| rs937219 | 0.0142 |
| rs2459731 | 0.0081 |
| rs576855 | 0.0034 |
| rs1187461 | 0.0142 |
| rs4701694 | 0.0165 |
| rs2617537 | −0.0241 |
| rs2652483 | 0.0083 |
| rs274667 | −0.0028 |
| rs274678 | −0.0022 |
| rs12514493 | 0.002 |
| rs340664 | −0.0068 |
| rs40687 | 0.0067 |
| rs1805962 | 0.0131 |
| rs194158 | −0.0381 |
| rs12520210 | 0.0203 |
| rs13174956 | 0.0235 |
| rs4263482 | 0.0093 |
| rs1015586 | 0.009 |
| rs10067241 | 0.0195 |
| rs4702826 | −0.023 |
| SNP_A- | −0.0141 |
| 1980768 | |
| rs730184 | 0.0137 |
| rs7706559 | 0.0124 |
| rs254833 | −0.0244 |
| rs2938443 | 0.0131 |
| rs4279307 | 0.0136 |
| rs1493475 | −0.0269 |
| rs4470727 | 0.0251 |
| rs12522491 | −0.003 |
| rs1428658 | −0.0355 |
| rs6895202 | 0.0313 |
| rs10051873 | 0.0276 |
| rs4291025 | −0.012 |
| rs16900210 | −0.0012 |
| rs12658519 | 0.0219 |
| rs7729075 | −0.0183 |
| rs2047725 | 0.0028 |
| rs630347 | −0.0138 |
| rs675048 | 0.0111 |
| rs6879013 | −0.0271 |
| rs659803 | 0.0243 |
| rs16890354 | −0.0386 |
| rs1158645 | −0.0249 |
| rs4703439 | 0.0315 |
| rs7701661 | 0.0198 |
| rs716971 | −0.0001 |
| rs3756468 | −0.0151 |
| rs17804894 | 0.0091 |
| rs2967213 | −0.0243 |
| rs962274 | −0.0308 |
| rs7379247 | −0.0011 |
| rs4866963 | −0.0063 |
| rs7448715 | 0.0143 |
| rs3846500 | −0.0013 |
| rs250210 | 0.0237 |
| rs9291972 | −0.0283 |
| rs6859355 | −0.0063 |
| rs35941 | −0.0141 |
| rs10043664 | −0.0142 |
| rs334879 | 0.0182 |
| rs6875155 | −0.0117 |
| rs158347 | −0.0208 |
| rs26714 | −0.0207 |
| rs37575 | 0.0083 |
| rs7714765 | 0.0249 |
| rs159358 | −0.0095 |
| rs6890351 | −0.0302 |
| rs4700577 | 0.0137 |
| rs10461494 | 0.0102 |
| rs6859021 | 0.0119 |
| rs7716822 | 0.0093 |
| rs1979015 | 0.021 |
| rs2561243 | −0.002 |
| rs1346533 | −0.0111 |
| rs1968378 | 0.0081 |
| rs963997 | −0.0052 |
| rs1370231 | −0.0014 |
| rs999826 | −0.01 |
| rs17307254 | −0.003 |
| rs501941 | −0.018 |
| rs11750626 | −0.0012 |
| rs343112 | −0.0326 |
| rs716707 | 0.0096 |
| rs11738470 | −0.0069 |
| rs6888419 | −0.0022 |
| rs3886043 | 0.0094 |
| rs6865628 | 0.0136 |
| rs10044370 | 0.0007 |
| rs10072221 | 0.0196 |
| rs17652917 | 0.0039 |
| rs17789758 | −0.0308 |
| rs9293725 | −0.0228 |
| rs2637076 | −0.0131 |
| rs2115436 | 0.0197 |
| rs1019803 | −0.0018 |
| rs236496 | −0.0058 |
| rs1366272 | 0.0264 |
| rs457700 | 0.0063 |
| rs10491244 | 0.0011 |
| rs224859 | −0.0208 |
| rs10076056 | 0.0082 |
| rs1022094 | 0.0176 |
| rs7721932 | 0.0022 |
| rs710377 | 0.0193 |
| rs4343874 | −0.0234 |
| rs648430 | 0.012 |
| rs666659 | −0.0017 |
| rs688956 | 0.0008 |
| rs9293546 | 0.0264 |
| rs4916813 | −0.0129 |
| rs825394 | 0.0345 |
| rs1862555 | −0.0065 |
| rs981862 | 0.0186 |
| rs2432175 | 0.0086 |
| rs149319 | 0.0087 |
| rs150822 | −0.0114 |
| rs436495 | −0.0227 |
| rs1009208 | 0.02 |
| rs13361147 | 0.0208 |
| rs12187983 | −0.001 |
| rs6880477 | 0.0019 |
| rs349323 | 0.0173 |
| rs10078913 | −0.0059 |
| rs7730302 | 0.0132 |
| rs2591425 | −0.0239 |
| rs12519534 | 0.0193 |
| rs1898510 | −0.0191 |
| rs295041 | −0.0308 |
| rs6888055 | −0.0002 |
| rs10515374 | 0.015 |
| rs6888461 | 0.0098 |
| rs1433057 | −0.005 |
| rs11240994 | −0.0082 |
| rs1378509 | −0.0372 |
| rs9326862 | −0.0425 |
| rs818428 | −0.0009 |
| rs7728185 | 0.0111 |
| rs7704624 | 0.0152 |
| rs6595016 | 0.0122 |
| rs10039799 | −0.0175 |
| rs7716581 | −0.0157 |
| rs1582931 | 0.0009 |
| rs10900767 | 0.0069 |
| rs6595443 | −0.0228 |
| rs4835799 | −0.0217 |
| rs1016475 | 0.01 |
| rs1017470 | 0.0249 |
| rs332042 | −0.01 |
| rs4836190 | −0.007 |
| rs4835907 | 0.0064 |
| rs2194079 | 0.0254 |
| rs3851469 | −0.0244 |
| rs10519977 | −0.0237 |
| rs13181926 | −0.0198 |
| rs17616306 | 0.0185 |
| rs2526195 | 0.003 |
| rs2577446 | 0.0049 |
| rs2577543 | 0.0202 |
| rs17616974 | 0.0149 |
| rs17616991 | 0.0216 |
| rs3798128 | 0.0241 |
| rs256215 | −0.0161 |
| SNP_A- | −0.0366 |
| 2244745 | |
| rs1006846 | −0.0162 |
| rs657223 | −0.0082 |
| rs703250 | −0.0211 |
| rs748355 | −0.0288 |
| rs4976402 | −0.0111 |
| rs2905591 | −0.0212 |
| rs825669 | 0.0019 |
| rs311596 | 0.0058 |
| rs825672 | 0.0193 |
| rs13160445 | −0.0313 |
| rs7704263 | 0.0146 |
| rs248496 | 0.0108 |
| rs33970 | 0.0047 |
| rs7710883 | 0.0037 |
| rs246425 | −0.022 |
| rs4357069 | 0.0395 |
| rs1549897 | −0.0015 |
| rs10037715 | 0.0131 |
| rs1981826 | −0.0042 |
| rs12521065 | −0.0029 |
| rs919724 | 0.0187 |
| rs246502 | 0.0333 |
| rs10491337 | 0.0025 |
| rs353297 | 0.0097 |
| rs17654574 | 0.015 |
| rs4705375 | 0.0116 |
| rs17461842 | −0.0075 |
| rs6898671 | −0.0154 |
| rs1864933 | 0.0182 |
| SNP_A- | −0.0158 |
| 1831010 | |
| rs3911203 | −0.001 |
| rs154965 | 0.0304 |
| rs17617120 | 0.0272 |
| rs17617422 | 0.0199 |
| rs253598 | −0.0191 |
| rs934418 | −0.0187 |
| rs10476244 | 0.0028 |
| rs12522186 | 0.0327 |
| rs210985 | 0.018 |
| rs211001 | 0.0202 |
| rs1458336 | −0.021 |
| rs883774 | 0.019 |
| rs17284960 | 0.0217 |
| rs262756 | 0.033 |
| rs17067598 | −0.0153 |
| rs4976578 | −0.0097 |
| rs7723533 | 0.0059 |
| rs2085449 | 0.0265 |
| rs4867939 | 0.0011 |
| rs297819 | −0.0203 |
| rs17667652 | −0.0432 |
| rs4867775 | −0.0184 |
| rs12716247 | 0.0183 |
| rs6879467 | −0.0081 |
| rs315730 | −0.0014 |
| rs2202440 | 0.0162 |
| rs838574 | 0.0148 |
| rs2964106 | 0.0018 |
| rs12513486 | 0.0107 |
| rs9503728 | −0.0001 |
| rs9328294 | −0.0152 |
| rs2432755 | 0.0128 |
| rs6909554 | −0.0116 |
| rs3804480 | −0.0262 |
| rs2064236 | 0.0252 |
| rs10900993 | −0.0278 |
| rs9504937 | 0.0066 |
| rs6927500 | −0.048 |
| rs1225933 | 0.0078 |
| rs7739573 | −0.0196 |
| rs11759202 | 0.0139 |
| rs7739767 | −0.0099 |
| rs6941454 | 0.0094 |
| rs6457228 | −0.0164 |
| rs4713981 | −0.0158 |
| rs9381033 | 0.0197 |
| rs2327544 | 0.0049 |
| rs2516234 | −0.0028 |
| rs9472528 | 0.0126 |
| rs204246 | −0.0121 |
| rs7766973 | −0.003 |
| rs1322753 | −0.0108 |
| rs9383046 | −0.031 |
| rs11757092 | 0.0375 |
| rs9476932 | −0.0021 |
| rs17580572 | 0.017 |
| rs2237164 | −0.0163 |
| rs12195984 | −0.0146 |
| rs6903672 | −0.0246 |
| rs9350045 | 0.0213 |
| rs2064187 | 0.0168 |
| rs1319785 | −0.0074 |
| rs9383403 | 0.0327 |
| SNP_A- | 0.0194 |
| 2071741 | |
| rs7739550 | −0.0035 |
| rs4712415 | −0.0133 |
| rs1319548 | −0.0166 |
| rs9358257 | −0.0117 |
| rs7752847 | 0.0062 |
| rs17205146 | −0.0068 |
| rs2005989 | −0.0278 |
| rs7751259 | 0.0238 |
| rs7743895 | −0.0243 |
| rs199750 | 0.0181 |
| rs10484434 | 0.0108 |
| rs2230655 | 0.0147 |
| rs198825 | −0.0068 |
| rs1233405 | 0.0317 |
| rs9357086 | −0.0092 |
| rs2844764 | −0.0045 |
| rs6933349 | 0.0225 |
| rs9263715 | −0.0021 |
| rs9263716 | 0.0039 |
| rs7761068 | 0.014 |
| rs3818527 | 0.0457 |
| rs11753875 | −0.0054 |
| rs3800472 | 0.0219 |
| rs969659 | −0.0158 |
| rs2237093 | 0.0161 |
| rs2654449 | 0.0076 |
| rs6908859 | 0 |
| rs9462481 | 0.0232 |
| rs1651110 | −0.0123 |
| rs1651106 | −0.0158 |
| rs1744416 | −0.0095 |
| rs11755763 | 0.0194 |
| rs7746124 | −0.0204 |
| rs7765472 | −0.0039 |
| rs1575764 | −0.0216 |
| rs9349145 | −0.0281 |
| rs6905110 | −0.0293 |
| rs9471589 | 0.0171 |
| rs4714560 | −0.0103 |
| rs9471810 | −0.0075 |
| rs9357446 | 0.0159 |
| rs7771912 | 0.0031 |
| rs9357499 | −0.0316 |
| rs910179 | 0.0105 |
| rs9472701 | −0.016 |
| rs614826 | −0.0048 |
| rs220701 | −0.0129 |
| rs6903181 | 0.0057 |
| rs10498774 | −0.0044 |
| rs2275446 | 0.0006 |
| rs7773311 | 0.0094 |
| rs4711923 | 0.0266 |
| rs12201810 | −0.0127 |
| rs6901180 | 0.0082 |
| rs2749029 | −0.0225 |
| rs3756982 | −0.0148 |
| rs9382181 | 0.0015 |
| rs2397146 | 0.0163 |
| rs9382239 | 0.0147 |
| rs4331986 | 0.0063 |
| rs4278019 | 0.0178 |
| rs10948783 | 0.0063 |
| rs10456715 | 0.0196 |
| rs3807016 | 0.0049 |
| rs4598117 | 0.0014 |
| rs12195233 | −0.0239 |
| rs10494880 | 0.0075 |
| rs10944779 | −0.0096 |
| rs6924505 | −0.01 |
| rs9363384 | −0.0292 |
| rs354369 | 0.0122 |
| rs902287 | −0.0118 |
| rs1938091 | −0.0081 |
| rs12202347 | 0.0043 |
| rs10806546 | 0.0058 |
| rs10944941 | 0.0285 |
| rs1454729 | −0.0124 |
| rs9342711 | 0.0237 |
| rs1204331 | −0.021 |
| rs2250128 | −0.0086 |
| rs7762984 | −0.023 |
| rs10805985 | 0.0146 |
| rs4235880 | 0.0201 |
| rs6454237 | −0.0065 |
| rs1145866 | −0.0258 |
| rs2048645 | 0.0025 |
| rs7767180 | 0.0063 |
| rs10943976 | −0.0022 |
| rs4527671 | −0.0045 |
| rs7742127 | 0.0028 |
| rs4706205 | 0.0035 |
| rs9362567 | −0.0079 |
| rs205199 | 0.0106 |
| rs2150817 | −0.017 |
| rs16883568 | 0.0036 |
| rs7750014 | −0.0084 |
| rs6924365 | 0.0079 |
| rs4299799 | 0.0202 |
| rs4235842 | 0.0213 |
| rs1535334 | 0.0165 |
| rs462846 | 0.0024 |
| rs4839985 | −0.0078 |
| rs550848 | 0.0323 |
| rs2894889 | 0.0101 |
| rs12182308 | −0.0048 |
| rs1996911 | −0.0036 |
| rs9391057 | 0.0277 |
| rs9377620 | 0.0095 |
| rs4945654 | 0.0237 |
| rs4946889 | −0.0254 |
| rs13210693 | 0.0265 |
| rs1111865 | 0.0055 |
| rs7752501 | 0.0291 |
| rs10872088 | −0.0033 |
| rs6919507 | 0.0148 |
| rs9387334 | −0.0074 |
| rs10484296 | 0.0218 |
| rs11153594 | −0.0197 |
| rs9481704 | −0.0008 |
| rs580435 | 0.0417 |
| rs465226 | 0.0077 |
| rs9489407 | −0.0192 |
| rs6941346 | 0.0007 |
| rs11154002 | 0.0073 |
| rs7738249 | 0.0139 |
| rs9490745 | −0.0173 |
| rs17085362 | −0.0103 |
| rs9385301 | −0.0216 |
| rs6931183 | −0.0187 |
| rs2626094 | 0.0189 |
| rs9385362 | −0.0229 |
| rs6569474 | 0.0297 |
| rs2608935 | −0.0052 |
| rs9493185 | −0.0188 |
| rs1590190 | 0.0094 |
| rs211591 | −0.0018 |
| rs6901981 | −0.0096 |
| rs11154829 | 0.0081 |
| rs1038214 | −0.0231 |
| rs9494756 | −0.0335 |
| rs7756251 | −0.0169 |
| rs10872474 | −0.0114 |
| rs2498644 | −0.0202 |
| rs9403045 | 0.0197 |
| rs1936215 | −0.0243 |
| rs2328092 | −0.0151 |
| rs2328165 | −0.0072 |
| rs9389952 | 0.0015 |
| rs12527836 | 0.0185 |
| rs6935203 | −0.037 |
| rs9390356 | −0.0019 |
| rs6930181 | 0.0247 |
| rs3924499 | 0.0091 |
| rs1572902 | −0.0024 |
| rs11155492 | −0.0046 |
| rs6915298 | 0.0005 |
| rs12214271 | −0.0305 |
| rs1339111 | 0.0057 |
| rs10484776 | −0.0275 |
| rs6940831 | −0.0218 |
| rs9383934 | 0.0018 |
| rs2982571 | −0.0089 |
| rs214944 | 0.0344 |
| rs1527369 | 0.0141 |
| rs1282444 | −0.0061 |
| rs684614 | 0.0332 |
| rs2169976 | −0.005 |
| rs7738498 | 0.0066 |
| rs9371823 | −0.0021 |
| rs6899710 | −0.0284 |
| rs9384361 | −0.0014 |
| rs9397869 | 0.0215 |
| rs2818064 | 0.0264 |
| rs1349890 | −0.0202 |
| rs9356125 | 0.0072 |
| rs4709424 | 0.0096 |
| rs294879 | 0.0085 |
| rs9347407 | 0.0125 |
| rs952901 | −0.0102 |
| rs2358 | 0.0192 |
| rs9356029 | 0.0114 |
| rs1399221 | 0.0114 |
| rs6455851 | −0.0051 |
| rs498325 | −0.0148 |
| rs3799630 | 0.0099 |
| rs13191136 | 0.0048 |
| rs6919615 | 0.0318 |
| rs1021544 | 0.0064 |
| rs2960839 | 0.028 |
| rs2056480 | −0.0249 |
| rs11772124 | 0.0045 |
| rs7786654 | −0.0087 |
| rs11773149 | −0.0002 |
| rs11766511 | −0.0094 |
| rs9886313 | 0.0089 |
| rs10272726 | −0.0126 |
| rs6959643 | 0.014 |
| rs37965 | −0.0127 |
| rs10256000 | −0.0139 |
| rs1017266 | 0.0045 |
| rs10952132 | 0.017 |
| rs7811128 | 0.0255 |
| rs2355756 | −0.0183 |
| rs11760715 | 0.0149 |
| rs1432492 | 0.0223 |
| rs12699509 | −0.0331 |
| rs36845 | 0.0038 |
| rs36856 | −0.0054 |
| rs6947830 | −0.0229 |
| rs12333630 | −0.0011 |
| rs11761152 | −0.0049 |
| rs6976624 | 0.007 |
| rs6461292 | 0.0028 |
| rs1403795 | −0.015 |
| rs2192486 | −0.0157 |
| rs985307 | 0.0047 |
| rs4721790 | 0.0091 |
| SNP_A- | 0.0211 |
| 4199965 | |
| rs1524102 | −0.0005 |
| rs10226018 | 0.0134 |
| rs6461717 | 0.0289 |
| rs39420 | −0.0025 |
| rs17152582 | 0.0251 |
| rs10215524 | 0.0236 |
| rs9639517 | 0.0285 |
| rs7810044 | 0.0208 |
| rs11980563 | 0.0372 |
| rs2014867 | −0.0081 |
| rs11763233 | 0.0222 |
| rs17723231 | −0.0136 |
| rs4509212 | −0.0247 |
| rs1419791 | 0.0256 |
| rs12530870 | 0.0021 |
| rs2302125 | −0.0097 |
| rs728585 | 0.01 |
| rs1029591 | 0.0067 |
| rs2330118 | 0.0181 |
| rs846334 | −0.0242 |
| rs846327 | −0.0239 |
| rs6463089 | −0.0087 |
| rs11771815 | 0.0131 |
| rs6959031 | 0.0371 |
| rs10274590 | −0.0293 |
| rs9649729 | −0.0007 |
| rs4917080 | 0.0003 |
| rs2237451 | −0.0176 |
| rs601899 | −0.0008 |
| rs603745 | 0.0001 |
| rs7796331 | 0.0298 |
| rs10499728 | −0.0014 |
| rs2030807 | −0.0155 |
| rs9642578 | −0.0193 |
| rs11974847 | 0.0193 |
| rs2706984 | −0.0065 |
| rs3886801 | −0.0034 |
| rs2642124 | 0.0129 |
| rs7794971 | 0 |
| rs4718862 | 0.0211 |
| rs1880368 | 0.0054 |
| rs10254839 | 0.0071 |
| rs10486881 | −0.0129 |
| rs3094901 | −0.0107 |
| rs6954820 | 0.0149 |
| rs12540244 | 0.0084 |
| rs7801657 | 0.0226 |
| rs479035 | 0.0138 |
| rs2960920 | 0.0095 |
| rs2855726 | −0.0097 |
| rs6971149 | 0.0223 |
| rs1636665 | −0.0075 |
| rs7778369 | 0.0227 |
| rs3801767 | −0.0039 |
| rs10272958 | −0.0206 |
| rs17160879 | −0.026 |
| rs10280549 | −0.0234 |
| rs31669 | 0.0008 |
| rs17161626 | −0.0002 |
| rs7801631 | 0.036 |
| rs2724355 | 0.0368 |
| rs1159658 | −0.009 |
| rs1916600 | 0.0137 |
| rs1357516 | 0.0128 |
| rs803271 | 0.0071 |
| rs2157814 | −0.0081 |
| rs11770288 | 0.0248 |
| rs854730 | 0.0036 |
| rs6465455 | 0.0049 |
| rs3917538 | −0.0399 |
| rs1053275 | 0.0412 |
| rs1682724 | −0.0199 |
| rs1524924 | 0.007 |
| rs17169479 | −0.0058 |
| rs1705107 | −0.0299 |
| rs995661 | −0.0302 |
| rs17269890 | −0.0104 |
| rs917274 | −0.0195 |
| rs176577 | −0.003 |
| rs7804699 | −0.0212 |
| rs3801948 | 0.0177 |
| rs2894475 | 0.0211 |
| rs2701685 | −0.0065 |
| rs13242621 | 0.0022 |
| rs17155196 | −0.0023 |
| rs12534073 | −0.0025 |
| rs17419000 | 0.0113 |
| rs1722053 | 0.0216 |
| SNP_A- | −0.0182 |
| 1931004 | |
| rs7802317 | −0.0086 |
| rs10499999 | −0.0132 |
| rs6980230 | −0.0027 |
| rs2074128 | −0.001 |
| rs2905286 | 0.0273 |
| rs12705856 | 0.0148 |
| rs2396625 | −0.0218 |
| rs10256129 | 0.0026 |
| rs10235236 | −0.0072 |
| rs727164 | 0.0182 |
| rs1525629 | 0.0132 |
| rs3779261 | −0.0101 |
| rs659416 | −0.0254 |
| rs600081 | 0.0095 |
| rs10081261 | 0.0203 |
| rs2060718 | 0.019 |
| rs6954433 | −0.007 |
| rs7811460 | −0.0205 |
| rs12707082 | 0.0051 |
| rs17167606 | 0.0207 |
| rs1563046 | −0.0209 |
| rs1488004 | −0.0074 |
| rs6467677 | −0.0071 |
| rs12671129 | 0.0196 |
| rs2465908 | −0.0161 |
| rs4363142 | 0.0255 |
| rs7780990 | 0.0171 |
| rs512927 | 0.0121 |
| rs7789244 | 0.009 |
| rs12703774 | −0.0196 |
| rs17162991 | 0.0202 |
| rs2011381 | 0.0073 |
| rs4726533 | −0.0216 |
| rs4726612 | 0.0057 |
| rs981969 | 0.0395 |
| rs9648818 | 0.0247 |
| rs7794745 | −0.0257 |
| rs10269376 | −0.0271 |
| rs2024266 | −0.0033 |
| rs2533146 | 0.0138 |
| rs1440455 | −0.0054 |
| rs1657257 | 0.0027 |
| rs1730173 | 0.0094 |
| rs10226143 | −0.0056 |
| rs1731837 | 0.0265 |
| rs2140629 | 0.0127 |
| rs10257910 | −0.0343 |
| rs2366559 | −0.0281 |
| rs10949693 | 0.021 |
| rs11777784 | −0.0156 |
| rs2469340 | 0.028 |
| rs2623595 | 0.0191 |
| rs11988752 | 0.0232 |
| rs1457179 | −0.0321 |
| rs2527760 | 0.0147 |
| rs1468391 | −0.0089 |
| rs17074879 | 0.0175 |
| rs2741110 | −0.0138 |
| rs2117616 | 0.0005 |
| rs1293288 | 0.0263 |
| rs4831366 | 0.0054 |
| SNP_A- | 0 |
| 1992337 | |
| rs11776929 | 0.0394 |
| SNP_A- | 0.0012 |
| 1950935 | |
| rs7812436 | 0.02 |
| rs13251640 | −0.0028 |
| rs1484799 | 0.0247 |
| rs10103576 | 0.0183 |
| rs11997729 | 0.0127 |
| rs2131227 | −0.0123 |
| rs2975183 | −0.028 |
| rs4872353 | −0.0114 |
| rs7813618 | 0.0005 |
| rs13280864 | 0.0061 |
| rs1429940 | 0.0158 |
| rs549371 | 0.0153 |
| rs519238 | 0.0256 |
| rs2726985 | −0.0187 |
| rs4733239 | 0.0143 |
| rs4236699 | −0.0007 |
| rs2716960 | 0.0027 |
| rs16878317 | 0.0206 |
| SNP_A- | −0.0365 |
| 2172528 | |
| rs16879344 | −0.0187 |
| rs16879773 | 0.0056 |
| rs13272876 | 0.0027 |
| rs4566997 | 0.0346 |
| rs4633028 | −0.0041 |
| rs6994035 | −0.0179 |
| rs4576415 | 0.0181 |
| rs4737164 | 0.0151 |
| rs3857964 | −0.0052 |
| rs6981587 | −0.0082 |
| rs10958703 | −0.0208 |
| rs11774804 | 0.0405 |
| rs13248571 | 0.0308 |
| rs1552380 | −0.0181 |
| rs442875 | −0.0259 |
| rs16917712 | 0.0109 |
| rs1989378 | 0.0304 |
| rs10808877 | −0.0206 |
| rs7001445 | 0.0129 |
| rs9643828 | 0.0156 |
| rs4737466 | 0.0219 |
| rs7006132 | 0.0189 |
| rs1375192 | 0.0152 |
| rs11987778 | −0.0233 |
| rs11985124 | −0.0322 |
| rs11986500 | −0.0124 |
| rs11787352 | 0.0317 |
| rs13273889 | 0.0342 |
| rs6988283 | 0.0405 |
| rs6415613 | −0.0103 |
| rs7833605 | −0.0121 |
| rs7815632 | −0.0195 |
| rs10504532 | 0.0349 |
| rs348180 | −0.0121 |
| rs1227639 | −0.0138 |
| rs9886374 | 0.0303 |
| rs2195099 | −0.0065 |
| rs10093631 | 0.0138 |
| rs4546659 | −0.0244 |
| rs2067807 | 0.0001 |
| rs10099886 | 0.0177 |
| rs10105026 | 0.0259 |
| rs6473536 | −0.0158 |
| rs10046686 | 0.022 |
| rs1427058 | 0.0352 |
| rs9692962 | 0.0043 |
| rs9297305 | −0.01 |
| rs218903 | 0.0144 |
| rs11780241 | −0.0198 |
| rs3847148 | −0.017 |
| rs3802191 | 0.0012 |
| rs1484584 | −0.0073 |
| rs12681330 | −0.0321 |
| rs2853277 | −0.0123 |
| rs1714402 | 0.0329 |
| rs4735498 | 0.0043 |
| rs2449550 | 0.0048 |
| rs1872016 | 0.0042 |
| rs1872017 | 0.002 |
| rs10504980 | 0.0211 |
| rs2154636 | 0.0301 |
| rs10099576 | −0.0235 |
| rs1269704 | 0.0164 |
| rs652008 | −0.018 |
| rs2511694 | −0.0283 |
| rs3098227 | 0.0281 |
| rs6992053 | 0.0063 |
| rs7003445 | −0.0064 |
| rs12546300 | −0.0099 |
| rs1672143 | −0.0066 |
| rs1583300 | 0.0154 |
| rs12544197 | −0.0229 |
| rs10091346 | 0.0008 |
| rs12386964 | −0.02 |
| rs11992996 | −0.0233 |
| rs2317380 | 0.0213 |
| rs218021 | −0.0066 |
| rs10094612 | −0.0248 |
| rs10103242 | −0.0266 |
| rs2468152 | −0.0331 |
| rs17828843 | 0.0073 |
| rs10113428 | −0.015 |
| rs4563917 | 0.0041 |
| rs2465373 | 0.0004 |
| rs2447183 | −0.0082 |
| rs2470025 | −0.0144 |
| rs1487173 | −0.0152 |
| rs2222459 | −0.0267 |
| rs4074890 | 0.0024 |
| rs1454618 | −0.0203 |
| rs2054066 | 0.0203 |
| rs6470435 | 0.0444 |
| rs4733601 | 0.0247 |
| rs2049825 | −0.0305 |
| rs2004375 | 0.0116 |
| rs10086626 | 0.0053 |
| rs1368700 | −0.0036 |
| rs7813092 | −0.0427 |
| rs12681074 | 0.0307 |
| SNP_A- | 0.014 |
| 1877597 | |
| rs2736862 | 0.0199 |
| rs7004093 | 0.0132 |
| rs11166894 | 0.006 |
| rs7830273 | 0.0093 |
| rs17565593 | 0.0082 |
| rs17624183 | 0.0178 |
| rs1949140 | −0.0192 |
| rs10096422 | 0.0139 |
| rs4637895 | −0.0133 |
| rs4909780 | −0.0267 |
| rs4387006 | −0.011 |
| rs16910015 | −0.0161 |
| rs13267587 | 0.0216 |
| rs4074243 | 0.0022 |
| rs16910231 | 0.0385 |
| rs7814156 | 0.0213 |
| rs2319425 | 0.0236 |
| rs9650577 | 0.0068 |
| rs2004243 | −0.0055 |
| rs2297083 | −0.0077 |
| rs4741213 | −0.0179 |
| rs1412252 | 0.0087 |
| rs10114509 | −0.0202 |
| rs1412696 | 0.0197 |
| rs7868524 | 0.0246 |
| rs635647 | 0.0077 |
| rs10974297 | −0.0088 |
| rs3895473 | −0.0192 |
| rs10122089 | 0.0074 |
| rs7859893 | 0.0335 |
| rs2773502 | −0.0175 |
| rs4742269 | 0.0264 |
| rs818889 | 0.0138 |
| rs9644892 | −0.0205 |
| rs10977865 | 0.002 |
| rs1408126 | −0.0166 |
| rs7863959 | −0.0146 |
| rs1330251 | −0.015 |
| rs1334045 | −0.0029 |
| rs1408808 | 0.021 |
| rs4741269 | 0.0217 |
| rs4741271 | 0.014 |
| rs10810093 | −0.0093 |
| rs17709614 | 0.0043 |
| rs10738434 | −0.0084 |
| rs10962370 | −0.0062 |
| rs1889007 | 0.0199 |
| rs10963742 | −0.0271 |
| rs10963810 | 0.009 |
| rs10964015 | −0.026 |
| rs10964496 | 0.0165 |
| rs7018694 | 0.0184 |
| rs10511679 | 0.002 |
| rs58024 | 0.0337 |
| rs7868378 | −0.0091 |
| rs10965244 | −0.02 |
| rs586554 | 0.0341 |
| rs10511712 | 0.0176 |
| rs1868727 | −0.001 |
| rs7465952 | 0.011 |
| rs932983 | −0.0323 |
| rs7046220 | −0.0294 |
| rs1231359 | 0.006 |
| rs10217376 | −0.0025 |
| rs12353404 | −0.0033 |
| rs763372 | −0.0029 |
| rs10968085 | −0.0215 |
| rs1625085 | −0.0351 |
| rs10812665 | −0.0159 |
| rs10968398 | 0.0215 |
| rs2996566 | 0.0073 |
| rs12351385 | 0.0177 |
| rs1328161 | 0.0122 |
| rs2674086 | 0.0359 |
| rs10970083 | 0.0152 |
| rs10970106 | −0.0338 |
| rs10813711 | 0.0015 |
| rs1061677 | −0.028 |
| rs927440 | −0.0208 |
| rs7038327 | −0.0063 |
| rs4880042 | −0.0077 |
| rs10814588 | 0.0132 |
| rs1467575 | −0.016 |
| rs7864204 | −0.0244 |
| rs11139084 | 0.0171 |
| rs1389124 | 0.0338 |
| rs10735607 | −0.0146 |
| rs11143049 | −0.0022 |
| rs17579544 | −0.0175 |
| rs3847322 | 0.0083 |
| rs11143575 | 0.0045 |
| rs10869430 | −0.0146 |
| rs10869435 | 0.0032 |
| rs1977240 | −0.0295 |
| rs1934735 | −0.0194 |
| rs10521467 | 0.0007 |
| rs2258131 | 0.0182 |
| rs2149589 | −0.0113 |
| rs526347 | −0.0234 |
| rs3739521 | −0.0036 |
| rs557352 | −0.0011 |
| rs11145021 | 0.0039 |
| rs9314853 | −0.0124 |
| rs7853550 | 0.0222 |
| rs10780227 | −0.0003 |
| rs7859620 | 0.034 |
| rs4384062 | −0.0073 |
| rs11139052 | −0.0101 |
| rs7047870 | 0.0338 |
| rs10117284 | 0.0171 |
| rs11140065 | −0.0101 |
| rs7037659 | 0.0076 |
| rs2111683 | −0.0146 |
| rs13294681 | −0.002 |
| rs10908927 | 0.0317 |
| rs7029450 | 0.0456 |
| rs1836406 | −0.0059 |
| rs1342140 | 0.0068 |
| rs12553207 | 0.0167 |
| rs2768279 | 0.0346 |
| rs6477581 | 0.0037 |
| rs7045958 | 0.0147 |
| rs2417801 | 0.0135 |
| rs1279850 | −0.0106 |
| rs4310281 | −0.0116 |
| rs10429635 | 0.0039 |
| rs1360291 | 0.0347 |
| rs10081687 | −0.0072 |
| rs7034422 | 0.0138 |
| rs1413410 | 0.0334 |
| rs7862181 | 0.0138 |
| rs16906169 | 0.0185 |
| rs7018934 | 0.008 |
| rs2151369 | −0.0283 |
| rs2416613 | 0.0204 |
| rs1999008 | −0.0162 |
| rs10818132 | −0.0144 |
| rs6478427 | 0.0055 |
| rs10818362 | 0.0058 |
| rs2151642 | 0.0141 |
| rs4837146 | −0.0284 |
| rs10901130 | 0.0167 |
| rs7861023 | −0.007 |
| rs783771 | 0.0132 |
| rs9411299 | −0.0282 |
| rs7085214 | 0.027 |
| rs11252693 | 0.027 |
| rs2804097 | −0.0221 |
| rs2805533 | −0.0275 |
| rs1909430 | 0.0248 |
| rs7100110 | 0.0204 |
| rs17182743 | 0.0021 |
| rs1325586 | 0.0012 |
| rs10795063 | −0.0205 |
| rs1885477 | 0.019 |
| rs10905668 | 0.0178 |
| rs10905669 | 0.021 |
| rs11254580 | 0.0048 |
| rs12416156 | 0.005 |
| rs4747792 | −0.0022 |
| rs10752138 | 0.0349 |
| rs2760206 | 0.0313 |
| rs17485180 | −0.0009 |
| rs7907578 | −0.0151 |
| rs10795771 | 0.0087 |
| rs7906212 | 0.0363 |
| rs727517 | 0.0202 |
| rs10906545 | −0.0006 |
| rs11259007 | 0.0243 |
| rs10906802 | −0.0083 |
| rs724830 | −0.0205 |
| rs10752073 | 0.0124 |
| rs12354883 | 0.0162 |
| rs7427 | −0.0073 |
| rs1414681 | −0.0248 |
| rs10828655 | −0.0045 |
| rs11014172 | 0.0259 |
| rs4415644 | −0.0168 |
| rs7897303 | −0.0106 |
| rs4749075 | −0.0249 |
| rs3006828 | 0.0407 |
| rs7087033 | 0.0234 |
| rs2478251 | −0.0076 |
| rs10763636 | −0.0167 |
| rs2249120 | −0.0173 |
| rs12254584 | 0.0002 |
| rs7910778 | 0.0131 |
| rs1328327 | −0.011 |
| rs1246762 | 0.0084 |
| rs6481677 | −0.0194 |
| rs598009 | 0.0089 |
| rs650788 | −0.0252 |
| rs17295289 | 0.0263 |
| rs2994655 | 0.0465 |
| rs4747738 | 0.0059 |
| rs11008296 | −0.0009 |
| rs793091 | 0 |
| rs806813 | −0.0154 |
| rs10827452 | 0.0067 |
| rs10827509 | 0.0072 |
| rs4244998 | 0.0053 |
| rs596815 | 0.0041 |
| rs708428 | 0.009 |
| rs2598575 | 0.0075 |
| rs2796780 | 0.0166 |
| rs2384598 | 0.0301 |
| rs10827687 | −0.0005 |
| rs2384682 | −0.0124 |
| rs2008449 | 0.0144 |
| rs189723 | −0.0024 |
| rs7917412 | −0.0326 |
| rs11239271 | −0.0173 |
| rs3860187 | −0.0252 |
| rs11101565 | 0.0168 |
| rs1880676 | 0.0163 |
| rs3810950 | 0.0205 |
| rs10824034 | 0.0095 |
| rs9651311 | 0.0032 |
| rs11000908 | 0.0037 |
| rs10508967 | 0.0087 |
| rs1413663 | −0.0046 |
| rs7913043 | −0.0077 |
| rs1360747 | −0.0147 |
| rs7088614 | 0.0075 |
| rs10825519 | −0.004 |
| rs1953304 | −0.0067 |
| rs1413668 | −0.0054 |
| rs1916521 | −0.006 |
| rs11005024 | 0.0154 |
| rs1361258 | 0.0063 |
| rs11005065 | 0.0052 |
| rs11005080 | 0.0041 |
| rs7100758 | 0.0308 |
| rs2393008 | −0.0035 |
| rs2393003 | −0.0021 |
| rs6481214 | 0.0133 |
| rs12780184 | 0.0285 |
| rs3910869 | −0.0068 |
| rs16913790 | −0.0154 |
| rs10821808 | 0.0136 |
| rs10761556 | 0.0101 |
| rs720600 | −0.014 |
| rs2448344 | −0.0137 |
| rs1906470 | 0.0223 |
| rs4314953 | −0.008 |
| rs7915131 | 0.0034 |
| rs1942006 | 0.0137 |
| rs17239782 | 0.0179 |
| rs1993183 | 0.0179 |
| rs7916781 | −0.0109 |
| rs3907196 | 0.0011 |
| rs10762030 | 0.0072 |
| rs3125312 | 0.0099 |
| rs10998022 | −0.0049 |
| rs9663508 | 0.0249 |
| rs11000666 | 0.0081 |
| rs7475340 | 0.0144 |
| rs2657291 | −0.0267 |
| rs1259598 | 0.0241 |
| rs11001720 | −0.0073 |
| rs2920792 | −0.0193 |
| rs10762732 | −0.0303 |
| rs10509384 | 0.0195 |
| rs1248571 | −0.0261 |
| rs11595067 | −0.009 |
| rs4980007 | −0.016 |
| rs1999990 | −0.0174 |
| rs10824657 | −0.0184 |
| rs2771236 | −0.0173 |
| rs2644209 | 0.0134 |
| rs7905007 | 0.0071 |
| rs4426079 | −0.0175 |
| rs12240734 | 0.0042 |
| rs11200819 | −0.0006 |
| rs7894012 | −0.0273 |
| rs7082134 | 0.0034 |
| rs4934353 | −0.0231 |
| rs7078618 | 0.0058 |
| rs812631 | −0.0099 |
| rs10887936 | −0.0064 |
| rs1120669 | −0.0076 |
| rs4933500 | −0.0055 |
| rs7087473 | 0.0303 |
| rs11186914 | −0.0157 |
| rs10450368 | 0.0155 |
| rs7068140 | 0.0101 |
| rs11187870 | 0.0058 |
| rs11188799 | 0.0093 |
| rs1502593 | −0.0357 |
| rs12357097 | −0.0185 |
| rs3781351 | 0.0262 |
| rs7087219 | 0.0202 |
| rs389094 | −0.0308 |
| rs12412977 | 0.0207 |
| rs10884820 | −0.0102 |
| rs11194926 | −0.0226 |
| rs7085888 | −0.007 |
| rs5015510 | −0.015 |
| rs10787328 | −0.008 |
| rs1547730 | −0.0371 |
| rs3781383 | 0.0111 |
| rs10787490 | 0.0141 |
| rs941853 | 0.0387 |
| rs10885719 | 0.0197 |
| rs17093112 | 0.0099 |
| rs406894 | −0.0145 |
| rs11198411 | −0.0288 |
| rs1556446 | 0.0314 |
| rs11199602 | −0.0035 |
| rs7911084 | −0.0107 |
| rs4751810 | −0.0039 |
| rs11598706 | 0.0086 |
| rs12774403 | 0.0219 |
| rs4131643 | 0.0282 |
| rs7078763 | 0.0184 |
| rs10901792 | 0.0033 |
| rs11245346 | −0.0044 |
| rs12768691 | 0.0087 |
| rs919831 | 0.0076 |
| rs10901402 | −0.0048 |
| rs10794008 | 0.0027 |
| rs10794069 | 0.0381 |
| rs7094049 | 0.0049 |
| rs11245205 | 0.006 |
| rs12359365 | −0.0115 |
| rs3740547 | −0.0258 |
| rs10764953 | −0.0049 |
| rs12416649 | 0.0216 |
| rs1655129 | −0.0018 |
| rs11017552 | −0.0134 |
| rs7075233 | −0.0395 |
| rs4897747 | 0.007 |
| rs2814184 | 0.0054 |
| rs6578397 | −0.0128 |
| rs17313216 | −0.0073 |
| rs1009240 | 0.0135 |
| rs2445335 | 0.0215 |
| rs4758441 | −0.011 |
| rs14229 | 0.0006 |
| rs4910363 | −0.0038 |
| rs10831786 | −0.0277 |
| rs2165484 | 0.0063 |
| rs4539308 | 0.0049 |
| rs4757128 | −0.0157 |
| rs10832000 | −0.0223 |
| rs1358560 | −0.0287 |
| rs12417170 | −0.0157 |
| rs4757001 | −0.0233 |
| rs2249878 | 0.0015 |
| rs1353649 | −0.0018 |
| rs10833522 | −0.0011 |
| rs11026573 | −0.0215 |
| rs10450560 | −0.0021 |
| rs10833888 | 0.0059 |
| rs7101766 | −0.0207 |
| rs2403995 | −0.0179 |
| rs12222611 | 0.0192 |
| rs293940 | 0.0293 |
| rs7943538 | 0.0126 |
| rs4547081 | 0.0254 |
| rs868607 | 0.0208 |
| rs12363459 | 0.0104 |
| rs7951928 | 0.019 |
| rs208107 | 0.0185 |
| rs1806156 | 0.0209 |
| rs10767929 | 0.0032 |
| rs2207549 | −0.0078 |
| rs4756695 | 0.0059 |
| rs10836202 | 0.0083 |
| rs704730 | 0.007 |
| rs1584807 | 0.0248 |
| rs7952315 | 0.0221 |
| rs4756190 | 0.029 |
| SNP_A- | 0.0476 |
| 4206828 | |
| rs11033972 | 0.0099 |
| rs1478767 | −0.0036 |
| rs10836978 | 0.0073 |
| rs11035666 | 0.0051 |
| rs2958851 | 0.0021 |
| rs7128465 | 0.0173 |
| rs995034 | −0.0242 |
| rs6485443 | −0.0335 |
| rs11037928 | −0.0188 |
| rs7926989 | 0.0186 |
| rs2863165 | 0.0132 |
| rs7951225 | −0.0233 |
| rs1052373 | 0.0115 |
| rs11040267 | 0.0129 |
| rs11040509 | −0.0034 |
| rs10897215 | 0.0067 |
| rs1675131 | 0.0286 |
| rs2236647 | −0.0053 |
| rs10895987 | 0.0002 |
| rs685469 | 0.013 |
| rs10793403 | 0.0224 |
| rs11235876 | 0.0244 |
| rs578477 | 0.0112 |
| rs2005145 | 0.0049 |
| rs7938883 | 0.0139 |
| rs7924613 | −0.0053 |
| rs12223477 | 0.0233 |
| rs7950813 | −0.038 |
| rs596737 | −0.0257 |
| rs549683 | −0.0134 |
| rs6591924 | 0.0341 |
| rs7121417 | −0.0071 |
| rs10897951 | −0.0058 |
| rs11233070 | −0.0093 |
| rs3912008 | −0.0114 |
| rs10431169 | 0.0082 |
| rs7117839 | 0.0513 |
| rs7479457 | −0.0288 |
| rs11019558 | 0.0326 |
| rs10741438 | 0.0117 |
| rs1379798 | 0.0156 |
| rs1703086 | 0.0422 |
| rs10830988 | −0.0015 |
| rs16918677 | −0.0116 |
| rs2045459 | 0.0121 |
| rs610611 | −0.0255 |
| rs586026 | −0.0035 |
| SNP_A- | −0.007 |
| 2258976 | |
| rs11021058 | −0.0039 |
| rs1943520 | 0.0081 |
| rs7938152 | −0.002 |
| rs10501838 | −0.015 |
| rs1690595 | 0.0217 |
| rs347759 | −0.0085 |
| rs7926001 | 0.0423 |
| rs5005274 | 0.002 |
| rs7107325 | 0.0321 |
| rs12419474 | 0.0151 |
| rs4754973 | 0.0058 |
| rs7397040 | −0.031 |
| rs477831 | −0.0178 |
| rs7107172 | 0.0219 |
| rs602625 | −0.0189 |
| rs11212408 | 0.023 |
| rs1789395 | −0.0099 |
| rs1648136 | 0.0005 |
| rs7105881 | −0.0015 |
| rs1268913 | −0.0137 |
| rs682155 | −0.0106 |
| rs2427651 | 0.0311 |
| rs4938659 | −0.0144 |
| rs1317815 | 0.0313 |
| rs11218032 | −0.0081 |
| rs11218552 | −0.0242 |
| rs17127202 | −0.0377 |
| rs653403 | −0.0169 |
| rs3923890 | 0.0176 |
| rs7952332 | 0.0077 |
| rs485327 | −0.0242 |
| rs2874773 | −0.0245 |
| rs1919267 | 0.0033 |
| rs10790928 | 0.0364 |
| rs7948351 | 0.0153 |
| rs476994 | 0.0248 |
| rs1065 | −0.0328 |
| rs11607783 | 0.0242 |
| rs4073610 | −0.0219 |
| rs4638317 | 0.0139 |
| rs498602 | 0.0124 |
| rs10458997 | 0.01 |
| rs880159 | 0.0091 |
| rs242032 | 0.0103 |
| rs11832578 | 0.018 |
| rs7965764 | 0.0087 |
| rs1015832 | −0.0331 |
| rs764220 | −0.0104 |
| rs11063875 | −0.0167 |
| rs10466913 | −0.0009 |
| rs7971637 | 0.0038 |
| rs4883421 | 0.0147 |
| rs4628755 | 0.0006 |
| rs4310684 | 0.0013 |
| rs1979268 | 0.013 |
| rs7963875 | 0.0136 |
| rs6488328 | 0.013 |
| rs2416946 | 0.0094 |
| rs1351227 | 0.0074 |
| rs2300242 | 0.0388 |
| rs1461039 | −0.0033 |
| rs1861595 | 0.0036 |
| rs17442423 | 0.0044 |
| rs1912879 | 0.0322 |
| rs6486975 | 0.0086 |
| rs10770712 | 0.0374 |
| rs4762694 | −0.0133 |
| rs7313671 | 0.0038 |
| rs10841833 | −0.0186 |
| rs2433643 | 0.0024 |
| rs10505944 | 0.0268 |
| rs11047761 | 0.0179 |
| rs859157 | −0.0192 |
| rs17337853 | −0.0145 |
| rs7954129 | −0.0124 |
| rs188551 | 0.0232 |
| rs6487856 | −0.0141 |
| rs7134917 | −0.0118 |
| rs7972251 | −0.0039 |
| rs7306223 | −0.0242 |
| rs1948034 | −0.0225 |
| rs4356315 | 0.0033 |
| rs11169482 | 0.0116 |
| rs4628717 | 0.0272 |
| rs7310430 | −0.0266 |
| rs12581243 | −0.0083 |
| rs7974787 | 0.0052 |
| rs1344828 | 0.0229 |
| rs11182168 | 0.0353 |
| rs11182199 | −0.0371 |
| rs12319024 | −0.0049 |
| rs9783418 | −0.0023 |
| rs4251466 | 0.014 |
| rs10880833 | 0.0312 |
| rs12322783 | 0.0087 |
| rs12818741 | 0.0087 |
| rs1995691 | −0.0181 |
| rs868884 | −0.018 |
| rs10783448 | −0.0155 |
| rs10783455 | −0.0149 |
| rs771998 | −0.0068 |
| rs10876222 | −0.0032 |
| rs10783486 | 0.0199 |
| rs10747650 | −0.0113 |
| rs17118865 | 0.0244 |
| rs12368653 | 0.0399 |
| rs3907516 | −0.022 |
| rs2712498 | 0.0037 |
| rs12230817 | 0.0091 |
| rs10784180 | 0.0182 |
| rs11173906 | 0.0048 |
| rs1245652 | 0.0143 |
| rs4093902 | −0.0139 |
| rs11174764 | 0.0187 |
| rs7977987 | −0.0188 |
| rs903088 | −0.0172 |
| rs7960495 | −0.0105 |
| rs2700146 | 0.0129 |
| rs1797707 | 0.0178 |
| rs2700078 | −0.0226 |
| rs10878749 | 0.0197 |
| rs7977634 | 0.0187 |
| rs12298772 | −0.007 |
| rs1584726 | 0.0009 |
| rs1240219 | 0.0087 |
| rs10748140 | −0.0189 |
| rs10784885 | 0.0028 |
| rs11178577 | −0.0114 |
| rs2956436 | 0.0152 |
| rs17111905 | −0.0109 |
| rs11180220 | −0.0122 |
| rs11180638 | 0.0183 |
| rs11104254 | −0.034 |
| rs10862334 | −0.0082 |
| rs7313736 | 0.0067 |
| rs17005594 | −0.0152 |
| rs7297808 | −0.0112 |
| rs7300188 | −0.0304 |
| rs10777020 | −0.0192 |
| rs796041 | −0.0266 |
| rs17458324 | −0.0013 |
| rs2520524 | 0.0187 |
| rs4842533 | 0.034 |
| rs10777370 | −0.0039 |
| rs337662 | −0.0128 |
| rs420271 | 0.002 |
| rs6538421 | 0.0156 |
| rs7486464 | 0.0092 |
| rs1345015 | −0.0187 |
| rs10860097 | 0.029 |
| rs4394881 | −0.0127 |
| rs7136406 | −0.0182 |
| rs7965985 | 0 |
| rs10860535 | 0.0095 |
| rs1488728 | −0.0196 |
| rs1488729 | 0.026 |
| rs7975540 | −0.0254 |
| rs35714 | −0.0159 |
| rs4764773 | 0.0133 |
| rs10745901 | 0.0113 |
| rs1607688 | 0.0032 |
| rs11111537 | −0.0169 |
| rs7295941 | 0.0198 |
| rs10778419 | 0.0323 |
| rs5009017 | −0.012 |
| rs10778619 | −0.0104 |
| rs2888967 | −0.0178 |
| rs10778624 | −0.0188 |
| rs1044994 | −0.035 |
| rs7952972 | −0.0058 |
| rs7309495 | 0.0018 |
| rs857747 | 0.0175 |
| rs6490034 | 0.0141 |
| rs7953468 | 0.0072 |
| rs540520 | −0.0164 |
| rs2292681 | 0.0311 |
| rs367997 | −0.0042 |
| rs7970937 | −0.0009 |
| rs10846924 | 0.0142 |
| rs4765122 | 0.0143 |
| rs2569817 | 0.008 |
| rs7967551 | −0.0095 |
| rs2699061 | 0.0058 |
| rs7957166 | −0.0172 |
| rs9788224 | 0.0182 |
| rs182046 | −0.0285 |
| rs4760022 | 0.0018 |
| rs4759642 | 0.0138 |
| rs9315472 | −0.0111 |
| rs9579838 | −0.0254 |
| rs3088071 | 0.0113 |
| rs480910 | −0.0333 |
| rs8181826 | 0.0256 |
| rs7991345 | 0.0144 |
| rs12427533 | −0.0246 |
| rs4770752 | −0.0071 |
| rs17082017 | 0.0066 |
| rs4770984 | −0.0116 |
| rs9319328 | 0.0136 |
| rs9512814 | −0.0248 |
| rs2093619 | 0.0335 |
| rs4110544 | −0.018 |
| rs11147381 | −0.0111 |
| rs590377 | 0.0055 |
| rs6490478 | 0.0142 |
| rs7333582 | 0.0152 |
| rs916732 | −0.0404 |
| rs2520704 | −0.0135 |
| rs206136 | 0.0082 |
| rs540109 | 0.0019 |
| rs523656 | −0.0149 |
| rs2858809 | −0.0149 |
| rs797212 | −0.0214 |
| rs7982817 | 0.017 |
| rs9315248 | 0.0263 |
| rs1578831 | −0.0376 |
| rs17821630 | 0.009 |
| rs9532047 | −0.0284 |
| rs2025411 | −0.0297 |
| rs957184 | 0.0154 |
| rs1186468 | 0.0219 |
| rs1028671 | −0.0172 |
| rs9315768 | −0.0284 |
| rs7323630 | 0.0199 |
| rs9532603 | 0.0089 |
| rs1887744 | −0.0031 |
| rs1328469 | 0.0001 |
| rs9594602 | −0.0102 |
| rs7994531 | 0.0015 |
| rs7334307 | 0.0003 |
| rs1044856 | 0.0025 |
| rs12870438 | 0.0085 |
| rs970691 | 0.0078 |
| rs4942447 | −0.0126 |
| rs9526114 | −0.0144 |
| rs1998891 | −0.001 |
| rs9534732 | −0.0018 |
| rs7322355 | 0.0022 |
| rs1951944 | 0.0032 |
| rs7984167 | 0.0033 |
| rs2181958 | −0.0196 |
| rs9534843 | 0.0065 |
| rs1966197 | −0.0001 |
| rs7317431 | −0.0286 |
| rs9568182 | 0.0265 |
| rs11619997 | 0.0115 |
| rs2066647 | −0.026 |
| rs1359614 | −0.0205 |
| rs6561620 | 0.0239 |
| rs9526823 | −0.0191 |
| rs9536222 | −0.025 |
| rs1360776 | −0.0331 |
| rs2000159 | 0.0186 |
| rs1341545 | 0.0146 |
| rs2592873 | −0.0138 |
| rs1410269 | −0.0151 |
| rs7986718 | −0.0162 |
| rs9538393 | −0.009 |
| rs965038 | −0.013 |
| rs1412973 | 0.0034 |
| rs9570966 | 0.0144 |
| rs2875357 | −0.0104 |
| rs12584386 | −0.0146 |
| rs1496154 | 0.0029 |
| rs4883781 | −0.0222 |
| rs8000469 | 0.0367 |
| rs9541348 | −0.0115 |
| rs1604614 | −0.0061 |
| rs9564577 | 0.0219 |
| rs9317873 | 0.0225 |
| rs9542388 | −0.006 |
| rs9529777 | 0.0014 |
| rs2769502 | −0.0105 |
| rs9599836 | 0.0057 |
| rs9529875 | 0.031 |
| rs9318105 | 0.0276 |
| rs9543259 | −0.0133 |
| rs9543742 | −0.0226 |
| rs9600484 | 0.0154 |
| rs17065026 | 0.0007 |
| rs9600671 | 0.0192 |
| rs1929734 | −0.0248 |
| rs1418774 | −0.0208 |
| rs9318496 | 0.0113 |
| rs1886480 | 0.0087 |
| rs9565375 | 0.0076 |
| rs9318512 | 0.0156 |
| rs1330881 | 0.0177 |
| rs7338981 | −0.0344 |
| rs1215445 | 0.0166 |
| rs9601461 | −0.025 |
| rs9634739 | 0.0223 |
| rs2497410 | −0.0168 |
| rs7984291 | 0.0152 |
| rs7323967 | −0.0146 |
| rs9559201 | −0.021 |
| rs8000348 | −0.0013 |
| rs9518907 | 0.0174 |
| rs11069505 | 0.0061 |
| rs1348292 | −0.015 |
| rs9586099 | 0.0156 |
| rs1855389 | −0.0023 |
| rs1472716 | 0.0168 |
| rs9516079 | −0.0075 |
| SNP_A- | 0.0002 |
| 2179996 | |
| rs9524382 | 0.0028 |
| rs17699419 | −0.0031 |
| rs2296999 | 0.0063 |
| rs4772218 | 0.0095 |
| rs11840709 | −0.0189 |
| rs6491631 | 0.0148 |
| rs9518494 | −0.0252 |
| rs7338828 | −0.0045 |
| rs6491882 | −0.0229 |
| rs9555135 | −0.0201 |
| rs7318970 | 0.0128 |
| rs12019929 | 0.0143 |
| rs11618877 | 0.0155 |
| rs9284246 | 0.0211 |
| rs1926532 | 0.0069 |
| rs1926530 | 0.0019 |
| rs1926528 | 0.0016 |
| rs558322 | −0.0218 |
| rs1411766 | 0.0112 |
| rs660433 | −0.0164 |
| rs9515279 | −0.0036 |
| rs12870443 | −0.0011 |
| rs9560075 | −0.0346 |
| rs9577771 | 0.0259 |
| rs9550117 | −0.0211 |
| rs3814260 | −0.0211 |
| rs10141075 | 0.0025 |
| rs1953558 | −0.0322 |
| rs12431912 | −0.0247 |
| rs12590446 | 0.0002 |
| rs11624594 | −0.0009 |
| rs222727 | −0.002 |
| rs1241649 | −0.009 |
| rs7144821 | 0.0014 |
| rs12880493 | 0.0112 |
| rs12880504 | 0.013 |
| rs10151818 | 0.0019 |
| rs1885036 | 0.0206 |
| rs2146631 | −0.0248 |
| rs4423349 | −0.0216 |
| rs7152910 | −0.0012 |
| rs8012040 | −0.0148 |
| rs1956404 | 0.0378 |
| rs10483377 | −0.0229 |
| rs10131977 | 0.0203 |
| rs10483369 | 0.0149 |
| rs915071 | 0.0259 |
| rs1952965 | 0.0381 |
| rs17477697 | 0.0267 |
| rs2383302 | 0.0118 |
| rs941746 | −0.012 |
| rs1957015 | 0.0176 |
| rs10147032 | 0.0114 |
| rs17520436 | −0.0482 |
| rs2149906 | −0.015 |
| rs6571668 | 0.0373 |
| rs7342504 | 0.0159 |
| rs7142040 | 0.0196 |
| rs12434601 | 0.0236 |
| rs10483469 | −0.0064 |
| SNP_A- | −0.0134 |
| 4289935 | |
| SNP_A- | −0.0134 |
| 2156181 | |
| rs7160534 | −0.0098 |
| rs2180090 | −0.0134 |
| rs1168519 | 0.0265 |
| rs8010051 | 0.0266 |
| rs17180580 | 0.0264 |
| rs365586 | 0.0238 |
| rs4903363 | −0.0044 |
| rs9707724 | 0.0054 |
| rs11625574 | −0.0049 |
| rs17615501 | 0.0004 |
| rs17115043 | 0.0169 |
| rs10139803 | −0.0077 |
| rs17700123 | 0.0075 |
| rs8022814 | −0.0076 |
| rs7160416 | −0.0124 |
| rs17121913 | 0.0036 |
| rs1869975 | 0.0182 |
| rs2181346 | 0.0222 |
| rs8012357 | −0.0005 |
| rs12431592 | 0.0021 |
| rs311841 | −0.0301 |
| rs4901870 | 0.0193 |
| rs1028897 | −0.0039 |
| rs17101715 | 0.0229 |
| rs4902293 | −0.016 |
| rs4902439 | −0.0102 |
| rs2478037 | −0.0058 |
| rs4899298 | −0.0198 |
| rs6574039 | 0.0265 |
| rs17119980 | 0.0129 |
| rs17182558 | −0.029 |
| rs887594 | −0.0313 |
| rs1548768 | 0.0003 |
| rs4148077 | 0.0291 |
| rs731952 | −0.0232 |
| rs2884739 | −0.0309 |
| rs10483891 | 0.0288 |
| rs6574590 | −0.02 |
| rs2372083 | 0.01 |
| rs17618463 | −0.0269 |
| rs12892711 | −0.009 |
| rs1958418 | 0.0067 |
| rs7158721 | 0.0068 |
| rs7150108 | −0.0153 |
| rs804951 | −0.0143 |
| rs4243678 | −0.0096 |
| rs17798191 | −0.03 |
| rs762018 | −0.0011 |
| rs8017825 | 0.0164 |
| rs373655 | 0.0154 |
| rs8008996 | 0.0191 |
| rs8007791 | 0.0183 |
| rs2749508 | −0.0177 |
| rs4900178 | 0.0193 |
| rs2144831 | −0.0311 |
| rs742897 | −0.0266 |
| rs881163 | −0.0241 |
| rs12896319 | 0.006 |
| rs730828 | −0.0166 |
| rs4905651 | −0.0296 |
| rs2199154 | −0.0269 |
| rs11621035 | 0.0095 |
| rs4905750 | 0.0125 |
| rs1022704 | −0.0202 |
| rs1388773 | −0.031 |
| rs11621256 | 0.0061 |
| rs1951570 | −0.0087 |
| rs6576461 | 0.018 |
| rs12915980 | −0.0223 |
| rs12910878 | −0.0298 |
| rs17646626 | −0.0111 |
| rs4778116 | 0.0116 |
| rs12915695 | −0.0015 |
| rs10519766 | −0.0056 |
| rs2082753 | 0.0186 |
| rs1864335 | −0.0125 |
| rs10152933 | −0.0264 |
| rs16961140 | −0.0331 |
| rs12905819 | 0.0225 |
| rs4244559 | 0.0049 |
| rs1520013 | 0.0046 |
| rs2929639 | −0.003 |
| rs2411241 | 0.0086 |
| rs8032900 | 0.0081 |
| rs2412456 | 0.0147 |
| rs1031963 | 0.0208 |
| rs6493004 | 0.0348 |
| rs12439488 | −0.0107 |
| rs579178 | 0.0102 |
| rs1484198 | 0.0128 |
| rs1426656 | −0.0117 |
| rs2663089 | 0.0033 |
| rs12148764 | 0.0145 |
| rs410665 | 0.0074 |
| rs4776125 | 0.0128 |
| rs2171476 | 0.008 |
| rs1906409 | −0.0034 |
| rs10518752 | 0.0037 |
| rs7172540 | 0.0313 |
| rs2414270 | −0.031 |
| rs4776222 | 0.0105 |
| rs11071129 | 0.0002 |
| rs12592527 | 0.0204 |
| rs935319 | 0.0083 |
| rs1280419 | −0.028 |
| rs11638057 | 0.0261 |
| rs536431 | −0.0069 |
| rs629876 | −0.0115 |
| rs1979422 | 0.02 |
| rs931971 | −0.0134 |
| rs7165960 | 0.0061 |
| rs7163836 | −0.0088 |
| rs9972449 | 0.0149 |
| rs3825957 | 0.0097 |
| rs4775349 | 0.0357 |
| rs8032386 | 0.0114 |
| rs990526 | −0.012 |
| rs955529 | −0.0325 |
| rs2292862 | 0.006 |
| rs982077 | 0.0115 |
| rs12907875 | −0.0067 |
| rs7497831 | −0.0029 |
| rs11634726 | 0.0233 |
| rs2114716 | 0.0023 |
| rs8029934 | 0.0166 |
| rs7169595 | 0.0023 |
| rs2343675 | −0.0154 |
| rs16940541 | −0.0015 |
| rs870689 | −0.0057 |
| rs8041327 | −0.0354 |
| rs1908786 | −0.0271 |
| rs16977786 | 0.0028 |
| rs11073678 | 0.0189 |
| rs4450366 | −0.006 |
| rs11073758 | −0.0089 |
| rs939590 | 0.0184 |
| rs11858077 | −0.0285 |
| rs293365 | −0.0114 |
| rs7164703 | −0.0013 |
| rs2074084 | −0.0051 |
| rs12437999 | 0.0345 |
| SNP_A- | 0.0238 |
| 1858077 | |
| rs12903400 | −0.0182 |
| rs11074097 | −0.0194 |
| rs2130100 | 0.0125 |
| rs16948232 | −0.0211 |
| rs7172568 | 0.0548 |
| rs1433943 | −0.0001 |
| rs11073398 | 0.0012 |
| rs7163584 | −0.0188 |
| rs172350 | −0.0087 |
| rs8036556 | −0.0114 |
| rs1036521 | −0.0057 |
| rs2654534 | −0.0145 |
| rs12910002 | 0.0171 |
| rs11631447 | 0.0147 |
| rs11630665 | 0.0211 |
| rs4334280 | −0.0093 |
| rs3923686 | 0 |
| rs11635298 | 0.0134 |
| rs8036374 | 0.0132 |
| rs11854332 | 0.0154 |
| rs3784482 | 0.036 |
| rs11649508 | −0.0277 |
| rs3760030 | 0.0151 |
| rs1919056 | 0.0417 |
| rs8057236 | −0.0065 |
| rs11643962 | −0.005 |
| rs9931343 | 0.0075 |
| rs1946127 | 0.0195 |
| rs17440477 | 0.0094 |
| rs1388807 | 0.0142 |
| rs2881438 | −0.0265 |
| rs7205378 | −0.0242 |
| rs11859000 | −0.0372 |
| rs12933673 | 0.0216 |
| rs2881629 | −0.0131 |
| rs4380041 | 0.0078 |
| rs9922185 | 0.0067 |
| rs7201849 | 0.0075 |
| rs1295741 | −0.0199 |
| rs2965860 | −0.0351 |
| rs17567906 | −0.0251 |
| rs2908710 | 0.0166 |
| rs7201430 | −0.0282 |
| rs7199390 | 0.0337 |
| rs11074996 | 0.0167 |
| rs1230896 | 0.0134 |
| rs2903305 | −0.0031 |
| rs2903308 | 0.01 |
| rs1428402 | 0.0052 |
| rs1050162 | 0.0099 |
| rs1840186 | −0.0144 |
| rs2764776 | 0.0009 |
| rs1014632 | 0.0061 |
| rs9922232 | 0.0151 |
| rs7499763 | 0.0104 |
| rs2521681 | 0.0173 |
| rs722516 | −0.0092 |
| rs6563855 | 0.0107 |
| rs3944008 | −0.0233 |
| rs9935379 | 0.0073 |
| rs7403957 | 0 |
| rs10521241 | −0.0146 |
| rs12599310 | −0.0465 |
| rs9940629 | 0.0225 |
| rs12149010 | 0.011 |
| rs4784376 | 0.023 |
| rs9929277 | −0.0051 |
| rs11863682 | −0.0103 |
| rs1809348 | 0.0045 |
| rs37358 | 0.01 |
| rs37363 | 0.0144 |
| rs2731740 | 0.0143 |
| rs9926973 | 0.0091 |
| rs2407755 | −0.0035 |
| rs7500302 | −0.001 |
| rs30888 | −0.0156 |
| rs1510212 | 0.0052 |
| rs2331446 | 0.003 |
| rs1126179 | −0.0022 |
| rs254363 | −0.0245 |
| rs11642497 | −0.0073 |
| rs1862667 | 0.0276 |
| rs1559307 | −0.0004 |
| rs1080383 | 0.0198 |
| rs7198357 | −0.0072 |
| rs7203768 | −0.01 |
| rs4889197 | −0.0026 |
| rs7194648 | 0.0013 |
| rs2866262 | −0.0037 |
| rs7185108 | −0.0074 |
| rs4528602 | 0.0079 |
| rs17688919 | 0.0182 |
| rs11646710 | 0.0107 |
| rs12917864 | −0.0095 |
| rs9925569 | 0.0171 |
| rs7197961 | 0.0198 |
| rs7202180 | −0.0049 |
| rs11649462 | 0.0205 |
| rs2967321 | 0.0124 |
| rs11647182 | 0.0181 |
| rs1424165 | 0.0106 |
| rs2042434 | −0.0039 |
| rs1035537 | −0.0055 |
| rs7195745 | −0.0138 |
| rs427976 | 0.0299 |
| rs3751797 | 0.0153 |
| rs4843404 | 0.0236 |
| rs4843405 | 0.0168 |
| rs8043893 | 0.0121 |
| rs8058785 | −0.0036 |
| rs4378618 | −0.0092 |
| rs7203610 | −0.0018 |
| rs6540223 | 0.0264 |
| rs3826067 | 0.0273 |
| rs2360735 | 0.0067 |
| SNP_A- | −0.0373 |
| 1832790 | |
| rs2131432 | −0.0183 |
| rs8072508 | 0.003 |
| SNP_A- | 0.0032 |
| 1833243 | |
| rs2955821 | 0.0105 |
| rs230404 | −0.0155 |
| rs150908 | 0.0194 |
| rs2567872 | 0.0032 |
| rs2567871 | 0.0008 |
| rs2325756 | 0.0132 |
| rs4262999 | 0.0192 |
| rs9908162 | −0.0283 |
| rs7214863 | 0.0151 |
| rs940854 | −0.0306 |
| rs4141200 | 0.0169 |
| rs4791362 | −0.0246 |
| rs7210608 | 0.0029 |
| rs4791489 | 0.015 |
| rs4791495 | −0.0291 |
| rs17560643 | 0.0305 |
| rs7212685 | 0.0223 |
| rs8068578 | −0.0228 |
| rs10852820 | −0.034 |
| rs1558267 | 0.0003 |
| rs956547 | −0.0182 |
| rs8080471 | −0.0003 |
| rs7212905 | 0.0051 |
| rs4393623 | −0.0063 |
| rs6502674 | 0.014 |
| rs8067882 | −0.003 |
| rs8069593 | 0.0108 |
| rs12943420 | 0.016 |
| rs6505282 | −0.0239 |
| rs16967400 | 0.0002 |
| rs7214958 | 0.0246 |
| rs121008 | −0.0079 |
| rs11655913 | −0.0011 |
| rs712046 | 0.024 |
| rs8074446 | 0.0276 |
| rs1109593 | 0.014 |
| rs8078723 | −0.0404 |
| rs2302777 | −0.005 |
| rs1405334 | 0.0348 |
| rs757274 | −0.0063 |
| rs9941364 | 0.0149 |
| rs757102 | 0.0062 |
| rs17658000 | −0.0081 |
| rs1378494 | −0.0071 |
| rs4796784 | 0.0242 |
| rs7222316 | −0.0072 |
| rs7209177 | 0.0033 |
| rs12601990 | −0.0208 |
| rs11713 | −0.0127 |
| rs4793035 | −0.0117 |
| rs4792814 | −0.0427 |
| rs1528072 | −0.0232 |
| rs7221510 | 0.0344 |
| rs7405452 | 0.0334 |
| rs757554 | −0.0187 |
| rs8064322 | 0.0115 |
| rs10515007 | 0.0232 |
| rs11079111 | 0.0015 |
| rs8072786 | −0.0251 |
| rs7501602 | −0.0204 |
| rs6504972 | 0.0181 |
| rs7220324 | 0.003 |
| rs4239197 | 0.0051 |
| rs8067664 | 0.0038 |
| rs8073334 | −0.0158 |
| rs7209070 | −0.0173 |
| rs2111016 | 0.0173 |
| rs870780 | −0.0213 |
| rs2585845 | 0.0097 |
| rs7223491 | 0.0208 |
| rs6504178 | 0.0063 |
| rs2665795 | −0.0018 |
| rs2727330 | 0.0052 |
| rs17688272 | −0.0189 |
| rs894582 | 0.0079 |
| rs12942924 | −0.008 |
| rs8073268 | 0.0201 |
| rs4791051 | −0.025 |
| rs7220127 | 0.0205 |
| rs7219499 | 0.0064 |
| rs9904266 | 0.006 |
| rs16959880 | 0.0048 |
| rs9910837 | 0.0268 |
| rs8073477 | 0.0116 |
| rs7210863 | 0.0021 |
| rs8067277 | 0.0019 |
| rs11652864 | −0.0031 |
| rs7226175 | 0.0101 |
| rs1093994 | −0.0257 |
| rs9911538 | −0.0277 |
| rs11077442 | −0.0239 |
| rs7216806 | −0.0155 |
| rs1046896 | 0.0032 |
| rs2846650 | 0.002 |
| rs7241142 | −0.0258 |
| rs9951259 | 0.0019 |
| rs568822 | −0.0031 |
| rs7506221 | −0.0178 |
| rs1658180 | −0.0077 |
| rs8095810 | −0.0158 |
| rs4797197 | 0.0098 |
| rs727616 | 0.0118 |
| rs17388230 | −0.0005 |
| rs4798520 | 0.0143 |
| rs1016188 | 0.0121 |
| rs1002715 | −0.007 |
| rs649598 | −0.0103 |
| rs619379 | 0.0168 |
| rs651568 | −0.0137 |
| rs12606001 | −0.0167 |
| rs4798749 | 0.0136 |
| rs633907 | 0.0133 |
| rs10502396 | −0.0051 |
| rs1874768 | 0.0094 |
| rs7231366 | −0.0333 |
| rs1284420 | −0.0053 |
| rs786038 | −0.0019 |
| rs786031 | −0.0056 |
| rs9957219 | 0.0194 |
| rs7229053 | 0.0117 |
| rs2852741 | 0.027 |
| rs573845 | −0.0172 |
| rs470463 | 0.0258 |
| rs16943923 | 0.0397 |
| rs11564410 | 0.0181 |
| rs9965582 | −0.0071 |
| rs1692481 | −0.0267 |
| rs356827 | 0.0096 |
| rs12964420 | 0.009 |
| rs1031732 | −0.024 |
| rs10502582 | −0.0262 |
| rs10502662 | 0.0132 |
| rs17651514 | −0.0219 |
| rs8090179 | −0.0167 |
| rs1905517 | −0.0125 |
| rs1367837 | −0.0021 |
| rs8096856 | −0.002 |
| rs4362464 | 0.0179 |
| rs9950114 | 0.0244 |
| rs12953383 | 0.0184 |
| SNP_A- | 0.02 |
| 4201660 | |
| rs11664621 | 0.0126 |
| rs9304346 | 0.0153 |
| rs299719 | 0.0142 |
| rs9952908 | 0.0382 |
| rs7235470 | 0.0164 |
| rs12955772 | 0.0201 |
| rs2953264 | −0.0146 |
| rs2953261 | 0.0078 |
| rs2879415 | −0.0038 |
| rs9962633 | 0.0206 |
| rs12960274 | −0.0219 |
| rs16957436 | −0.0276 |
| rs969047 | 0.0144 |
| rs1558536 | −0.0132 |
| rs9959800 | 0.0145 |
| rs4801058 | −0.0223 |
| rs8094838 | −0.0236 |
| rs597741 | 0.0028 |
| rs3865419 | 0.0145 |
| rs4058288 | 0.015 |
| rs12454023 | 0.0118 |
| rs12457509 | −0.0088 |
| rs3744865 | −0.016 |
| rs2163107 | 0.0148 |
| rs11665249 | 0.0192 |
| rs4368253 | 0.0231 |
| rs650313 | 0.0117 |
| rs8099832 | 0.012 |
| rs8083437 | 0.0091 |
| rs1118433 | 0.007 |
| rs9961166 | 0.0153 |
| rs950408 | −0.0124 |
| rs17665435 | 0.0325 |
| rs683109 | 0.0029 |
| rs3810031 | 0.0235 |
| rs2850761 | −0.0012 |
| rs7227145 | −0.0151 |
| rs1455557 | 0.0023 |
| rs17071220 | −0.0228 |
| rs1863583 | 0.0042 |
| rs13370501 | 0.0112 |
| rs2715282 | 0.0198 |
| rs2333505 | −0.0023 |
| rs8088426 | −0.0116 |
| rs8088774 | −0.016 |
| rs11151403 | 0.0074 |
| rs7226844 | −0.0139 |
| rs12960357 | 0.0208 |
| rs8096331 | −0.0164 |
| rs4426448 | 0.0455 |
| SNP_A- | 0.0246 |
| 1836916 | |
| rs17082947 | 0.0204 |
| rs4243328 | 0.0161 |
| rs17225992 | −0.0124 |
| rs1879682 | 0.0058 |
| rs2685441 | −0.0292 |
| rs1942474 | −0.0077 |
| rs12965641 | −0.0057 |
| rs8094131 | 0.003 |
| rs8089151 | 0.0346 |
| rs7407812 | 0.0091 |
| rs11875775 | 0.0155 |
| rs690227 | −0.0028 |
| rs10514217 | −0.0081 |
| rs1717548 | 0.003 |
| rs1623269 | −0.0139 |
| rs610806 | −0.0139 |
| rs3809928 | 0.0028 |
| rs12606550 | −0.001 |
| rs7260635 | −0.0022 |
| rs415647 | −0.0163 |
| rs2216662 | 0.0143 |
| rs1862462 | 0.0115 |
| rs2431795 | 0.0122 |
| rs3745597 | −0.0288 |
| rs7256992 | 0.045 |
| rs12710152 | 0.011 |
| rs12982420 | 0.0263 |
| rs11673570 | −0.0145 |
| rs11879734 | 0.0039 |
| rs12609496 | −0.0071 |
| rs7249829 | 0.0199 |
| rs33439 | 0.0336 |
| rs3745785 | −0.0104 |
| rs7258461 | 0.0134 |
| rs16967984 | −0.016 |
| rs285686 | 0.0239 |
| rs10421478 | −0.0208 |
| rs10414846 | 0.0202 |
| rs1402468 | 0.0144 |
| rs1620082 | −0.0258 |
| rs7248248 | 0.0036 |
| rs11083515 | 0.0133 |
| rs2159324 | 0.0109 |
| rs4802322 | −0.0064 |
| rs12461370 | −0.0125 |
| rs2303690 | −0.0106 |
| rs10404905 | 0.0248 |
| rs753307 | 0.0103 |
| rs10401904 | 0.0012 |
| rs10411879 | −0.0189 |
| rs8105809 | −0.0001 |
| rs9941465 | −0.0313 |
| rs302838 | 0.0393 |
| rs8108715 | −0.0146 |
| rs7252632 | −0.0349 |
| rs10408146 | −0.0114 |
| rs10417057 | 0.0111 |
| rs6118784 | −0.005 |
| rs6133763 | 0.001 |
| rs2143541 | −0.0059 |
| rs1161237 | −0.0007 |
| rs1178015 | −0.015 |
| rs6115865 | −0.0338 |
| rs241601 | 0.0096 |
| rs6139317 | −0.0085 |
| rs6052673 | 0.0034 |
| rs3746674 | 0.0328 |
| rs1343178 | −0.0196 |
| rs990928 | 0.0206 |
| rs1569874 | 0.0103 |
| rs6056909 | 0.0215 |
| rs6074180 | −0.018 |
| rs6134481 | −0.0066 |
| rs6041229 | −0.0169 |
| rs6074548 | −0.0032 |
| rs6033574 | −0.002 |
| rs8183260 | −0.011 |
| rs6042376 | −0.0378 |
| rs3932489 | 0.0212 |
| rs1225888 | 0.0114 |
| rs6080193 | −0.0161 |
| rs2208766 | −0.0211 |
| rs753213 | 0 |
| rs6075279 | −0.0046 |
| rs16979106 | 0.0018 |
| rs1475126 | 0.0154 |
| rs6081489 | 0.0253 |
| rs6035224 | 0.0001 |
| rs1555271 | 0.0178 |
| rs199790 | −0.0099 |
| rs199793 | −0.012 |
| rs16984335 | 0.0107 |
| rs7353712 | 0.022 |
| rs2260455 | 0.0149 |
| rs6050403 | −0.0307 |
| rs1543438 | −0.0068 |
| rs6088619 | 0.0048 |
| rs6060669 | 0.0399 |
| rs8116328 | −0.0164 |
| rs1205441 | −0.0041 |
| rs1119803 | −0.0166 |
| rs4811821 | −0.0082 |
| rs1002154 | −0.0049 |
| rs4810281 | 0.0086 |
| rs941797 | 0.0214 |
| rs4812566 | 0.0135 |
| rs2223542 | 0.0027 |
| rs8119733 | −0.0035 |
| rs6103041 | 0.034 |
| rs1973949 | −0.0078 |
| rs927058 | 0.0168 |
| rs459681 | −0.0058 |
| rs6032544 | 0.0068 |
| rs376438 | 0.0057 |
| rs6019032 | 0.0119 |
| rs2426103 | 0.0223 |
| rs660613 | 0.0178 |
| rs4591400 | 0.0216 |
| rs11906291 | 0.0014 |
| rs230002 | 0.0132 |
| rs6021700 | 0.0133 |
| rs4809901 | −0.0063 |
| rs6013463 | −0.0121 |
| rs1403742 | −0.0089 |
| rs2256452 | −0.022 |
| rs6022784 | 0.0181 |
| rs4811614 | 0.0151 |
| rs6024333 | 0.0061 |
| rs6070149 | −0.023 |
| rs6026574 | −0.0104 |
| rs13037047 | 0.0134 |
| rs2427144 | −0.0112 |
| rs2822524 | 0.003 |
| rs2822891 | −0.0221 |
| rs2822892 | −0.0191 |
| rs412399 | 0.0124 |
| rs420571 | −0.0047 |
| rs12482230 | 0.0082 |
| rs2823806 | 0.0021 |
| rs414663 | −0.016 |
| rs11088618 | −0.0186 |
| rs2776064 | −0.0287 |
| rs17777477 | −0.0173 |
| rs1079827 | 0.0015 |
| SNP_A- | −0.002 |
| 1969384 | |
| rs9981165 | 0.0297 |
| rs2827528 | −0.0248 |
| rs2827915 | 0.0091 |
| rs1506009 | −0.0251 |
| rs2262265 | 0.0002 |
| rs1892728 | −0.0353 |
| rs465318 | −0.009 |
| rs3017533 | 0.0204 |
| rs235930 | −0.0109 |
| rs2156292 | 0.0151 |
| rs2830986 | 0.0113 |
| rs2831543 | −0.0048 |
| rs383576 | −0.0105 |
| rs363487 | −0.025 |
| rs1016700 | −0.009 |
| rs2832445 | −0.0201 |
| rs2252898 | −0.0045 |
| rs2833423 | −0.0116 |
| rs2833538 | −0.015 |
| rs8134837 | −0.019 |
| rs2012993 | 0.0082 |
| rs2833654 | −0.0379 |
| rs933122 | −0.0011 |
| rs2070359 | −0.0148 |
| rs7280071 | 0.011 |
| rs4817889 | 0.0221 |
| rs2836482 | 0.0157 |
| rs1022423 | −0.0266 |
| rs1235555 | 0.0333 |
| rs7282356 | 0.015 |
| rs746063 | 0.0269 |
| rs2838008 | 0.0088 |
| rs9305744 | 0.0091 |
| rs4346474 | 0.0081 |
| rs8132937 | −0.0077 |
| rs690260 | −0.0366 |
| rs4819962 | 0.0286 |
| rs5747103 | −0.0138 |
| rs3827281 | −0.0002 |
| rs5748015 | −0.0076 |
| rs2800973 | 0.0116 |
| rs741194 | −0.003 |
| rs5997062 | −0.0227 |
| rs1000815 | −0.0133 |
| rs2213758 | −0.0196 |
| rs5997178 | −0.0247 |
| rs5997228 | −0.0323 |
| rs761594 | 0.0453 |
| rs2065057 | −0.0147 |
| rs926335 | −0.0122 |
| rs239327 | 0.0213 |
| rs130707 | −0.019 |
| rs9607337 | −0.0335 |
| rs131838 | −0.0108 |
| rs2284060 | 0.008 |
| rs738477 | 0.0066 |
| rs738479 | −0.0248 |
| rs12628020 | −0.0221 |
| rs5765532 | −0.0332 |
| rs5768257 | 0.0205 |
| rs5766999 | 0.0136 |
| rs5770018 | −0.0107 |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in genetics, diagnostic assays, molecular biology or related fields are intended to be within the scope of the following claims.

1. A method for diagnosing an autism spectrum disorder (ASD), or predisposition to develop an ASD, in a subject, which comprises the step of investigating a set of single nucleotide polymorphisms (SNPs) in a sample from the subject, wherein the number of SNPs in the set is such that the method can diagnose ASD with at least 70% accuracy.

2. A method according to claim 1, wherein the set comprises at least 1500 SNPs from the list given in Table 3.

3. A method according to claim 2, wherein the set comprises at least 2300 SNPs from the list given in Table 3.

4. A method according to claim 2, wherein the set comprises all 3126 SNPs given in Table 3.

5. A method according to claim 1, wherein the set comprises at least 70% of the SNPs weighted at least ±0.01 in Table 3.

6. A method according to claim 1, wherein the set comprises between 1500 and 4500 SNPs.

7. A method according to claim 1, wherein the set comprises between 2300 and 3900 SNPs.

8. A method according to claim 1, wherein the set comprises between 3000 and 3300 SNPs.

9. A kit for diagnosing an autism spectrum disorder (ASD), or predisposition to develop an ASD, in a subject, which comprises a plurality of primer pairs or probes capable of investigating a set of single nucleotide polymorphisms (SNPs) in a sample from the subject, wherein the set of SNPs comprises at least 1500 SNPs from the list given in Table 3.

10. A kit according to claim 9, wherein the plurality of probes is immobilised on a solid support.

11. A method for preparing a kit according to claim 10 which comprises the step of immobilising the plurality of probes on to a solid support.
